(19) United States
(12) Patent Application Publication
(10) Pub. No.: US 2017/0077415 A1
(43) Pub. Date: Mar. 16, 2017

(54) AN ELECTRON TRANSPORT MATERIAL AND AN ORGANIC ELECTROLUMINESCENCE DEVICE COMPRISING THE SAME

(71) Applicant: Rohm and Haas Electronic Materials Korea Ltd., Cheonan (KR)

(72) Inventors: Chi-Sik Kim, Hwaseong (KR); Ji-Song Jun, Hwaseong (KR); Young-Jun Cho, Seongnam (KR); Kyung-Hoon Choi, Hwaseong (KR); Sang-Hee Cho, Suwon (KR); Jae-Hoon Shim, Seoul (KR); Hong-Yeop Na, Seoul (KR)

(21) Appl. No.: 15/309,233

(22) PCT Filed: May 8, 2015
(86) PCT No.: PCT/KR2015/004640

§ 371 Date: Nov. 7, 2016

(30) Foreign Application Priority Data
May 8, 2014 (KR) 10-2014-0055106
May 7, 2015 (KR) 10-2015-0063687

(51) Int. Cl. H01L 51/00; C07D 409/14; C07D 403/14; C07D 405/14; C07D 403/04; C07D 403/10

(52) U.S. Cl. CPC H01L 51/0067; C07D 403/04; H01L 51/0072; C07D 403/10; H01L 51/0052; C07D 403/14; C07D 405/14; H01L 51/0073; C07D 409/14; H01L 51/0074; H01L 51/5072

(57) ABSTRACT

The present invention relates to an organic electroluminescent device comprising an electron transport material which comprises a compound having a specific structure. The organic electroluminescent device comprising the electron transport material of the present invention provides low driving voltage, high luminous efficiency, excellent lifespan characteristics, and excellent color coordination to efficiently emit blue light.

### TECHNICAL FIELD

The present invention relates to an electron transport material having improved electron transport ability, and an organic electroluminescent device comprising the same.

### BACKGROUND ART

An electroluminescent (EL) device is a self-light-emitting device with the advantages of providing a wider viewing angle, a greater contrast ratio, and a faster response time. The first organic EL device was developed by Eastman Kodak, by using small aromatic diamine molecules and aluminum complexes as materials for forming a light-emitting layer (see Appl. Phys. Lett. 51, 913, 1987).

An organic EL device changes electric energy into light by the injection of a charge into an organic light-emitting material, and commonly comprises an anode, a cathode, and an organic layer formed between the two electrodes. The organic layer of the organic EL device may be composed of a hole injection layer (HIL), a hole transport layer (HTL), an electron blocking layer (EBL), a light-emitting layer (EML) (containing host and dopant materials), an electron buffer layer, a hole blocking layer (HBL), an electron transport layer (ETL), an electron injection layer (EIL), etc.; the materials used in the organic layer can be classified into a hole injection material, a hole transport material, an electron blocking material, a light-emitting material, an electron buffer material, a hole blocking material, an electron transport material, an electron injection material, etc., depending on functions. In the organic EL device, holes from an anode and electrons from a cathode are injected into a light-emitting layer by electric voltage, and an exciton having high energy is produced by the recombination of the holes and electrons. The organic light-emitting compound moves into an excited state by the energy and emits light from energy when the organic light-emitting compound returns to the ground state from the excited state.

The most important factor determining luminous efficiency in an organic EL device is light-emitting materials. The light-emitting materials are required to have the following features: high quantum efficiency, high movement degree of an electron and a hole, and uniformality and stability of the formed light-emitting material layer. The light-emitting material is classified into blue, green, and red light-emitting materials according to the light-emitting color, and further includes yellow or orange light-emitting materials. Furthermore, the light-emitting material is classified into a host material and a dopant material in a functional aspect. Recently, an urgent task is the development of an organic EL device having high efficiency and long lifespan. In particular, the development of highly excellent light-emitting material over conventional materials is urgently required, considering the EL properties necessary for medium- and large-sized OLED panels. For this, preferably, as a solvent in a solid state and an energy transmitter, a host material should have high purity and a suitable molecular weight in order to be deposited under vacuum. Furthermore, a host material is required to have high glass transition temperature and pyrolysis temperature for guaranteeing thermal stability, high electrochemical stability for long lifespan, easy formability of an amorphous thin film, good adhesion with adjacent layers, and no movement between layers.

Meanwhile, in an organic EL device, an electron transport material actively transports electrons from a cathode to a light-emitting layer and inhibits transport of holes which are not recombined in the light-emitting layer to increase recombination opportunity of holes and electrons in the light-emitting layer. Thus, electron-affinitive materials are used as an electron transport material. Organic metal complexes having light-emitting function such as Alq₃ are excellent in transporting electrons, and thus have been conventionally used as an electron transport material. However, Alq₃ has problems in that it moves to other layers and shows reduction of color purity when used in blue light-emitting devices. Therefore, new electron transport materials have been required, which do not have the above problems, are highly electron-affinitive, and quickly transport electrons in organic EL devices to provide organic EL devices having high luminous efficiency.

Korean Patent Appin. Laying-Open No. KR 2010-0130197 A discloses a compound wherein a nitrogen-containing heterocycle is bonded to a nitrogen atom of an indenocarbazole backbone. However, it fails to disclose an organic EL device using the compound as an electron transport material.

The present inventors found that high efficiency and long lifespan of an organic EL device are provided when using a compound of a specific structure having an indenocarbazole backbone wherein a nitrogen atom of the carbazole is bonded to a nitrogen-containing heterocycle as an electron transport material.

### DISCLOSURE OF THE INVENTION

### Problems to be Solved

The objective of the present invention is to provide an electron transport material which can produce an organic EL device having high efficiency and long lifespan.

### Solution to Problems

The above objective can be achieved by an electron transport material comprising the compound represented by the following formula 1:

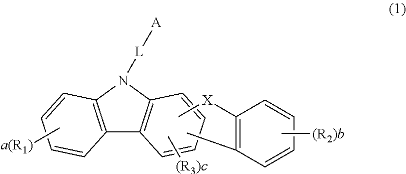

wherein

A represents a substituted or unsubstituted 5- to 30-membered heteroaryl;

L represents a single bond, a substituted or unsubstituted (C6-C30)arylene, or a substituted or unsubstituted 5- to 30-membered heteroarylene;

X represents CR₁₁R₁₂;

R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring whose carbon atom(s) may be replaced with at least one hetero atom selected from nitrogen, oxygen, and sulfur;

R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring whose carbon atom(s) may be replaced with at least one hetero atom selected from nitrogen, oxygen, and sulfur;

R₁₁ and R₁₂ each independently represent a substituted or unsubstituted (C1-C30)alkyl, a substituted or unsubstituted (C6-C30)aryl, or a substituted or unsubstituted 5- to 30-membered heteroaryl; or are linked to each other to form a mono- or polycyclic (C3-C30) alicyclic or aromatic ring whose carbon atom(s) may be replaced with at least one hetero atom selected from nitrogen, oxygen, and sulfur;

a and b each independently represent an integer of 1 to 4, where a or b is an integer of 2 or more, each of R₁ and each of R₂ may be the same or different;

c represents an integer of 1 to 2, where c is 2, each of R₃ may be the same or different; and

the heteroaryl(ene) contains at least one hetero atom selected from B, N, O, S, P(═O), Si, and P.

### Effects of the Invention

By using the electron transport material according to the present invention, an organic EL device with high efficiency and long lifespan is provided, and it is possible to produce a display device or a lighting device using the organic EL device.

### BRIEF DESCRIPTION OF THE FIGURES

FIG. 1 illustrates a schematic sectional view of an organic electroluminescent device comprising the electron transport layer comprising the electron transport material according to one embodiment of the present invention.

FIG. 2 illustrates a comparison of current efficiency between an organic electroluminescent device according to one embodiment of the present invention and a conventional organic electroluminescent device.

FIG. 3 illustrates an energy gap relationship among the layers of the organic electroluminescent device according to one embodiment of the present invention.

### EMBODIMENTS OF THE INVENTION

Hereinafter, the present invention will be described in detail. However, the following description is intended to explain the invention, and is not meant in any way to restrict the scope of the invention.

Hereinafter, the compound represented by formula 1 will be described in detail.

Herein, “(C1-C30)alkyl” is meant to be a linear or branched alkyl having 1 to 30 carbon atoms, in which the number of carbon atoms is preferably 1 to 10, more preferably 1 to 6, and includes methyl, ethyl, n-propyl, isopropyl, n-butyl, isobutyl, tert-butyl, etc. “(C2-C30)alkenyl” is meant to be a linear or branched alkenyl having 2 to 30 carbon atoms, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc. “(C2-C30)alkynyl” is a linear or branched alkynyl having 2 to 30 carbon atoms, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc. “(C3-C30)cycloalkyl” is a mono- or polycyclic hydrocarbon having 3 to 30 carbon atoms, in which the number of carbon atoms is preferably 3 to 20, more preferably 3 to 7, and includes cyclopropyl, cyclobutyl, cyclopentyl, cyclohexyl, etc. “3- to 7-membered heterocycloalkyl” is a cycloalkyl having at least one heteroatom selected from B, N, O, S, P(═O), Si, and P, preferably O, S, and N, and 3 to 7 ring backbone atoms, and includes tetrahydrofuran, pyrrolidine, thiolan, tetrahydropyran, etc. “(C6-C30)aryl(ene)” is a monocyclic or fused ring derived from an aromatic hydrocarbon having 6 to 30 carbon atoms, in which the number of carbon atoms is preferably 6 to 20, more preferably 6 to 15, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc. “5- to 30-membered heteroaryl(ene)” is an aryl group having at least one, preferably 1 to 4 heteroatoms selected from the group consisting of B, N, O, S, P(═O), Si, and P, and 5 to 30 ring backbone atoms; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzonaphthothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. “Halogen” includes F, Cl, Br, and I.

The compound of formula 1 may be represented by one of the following formulae 2 to 7:

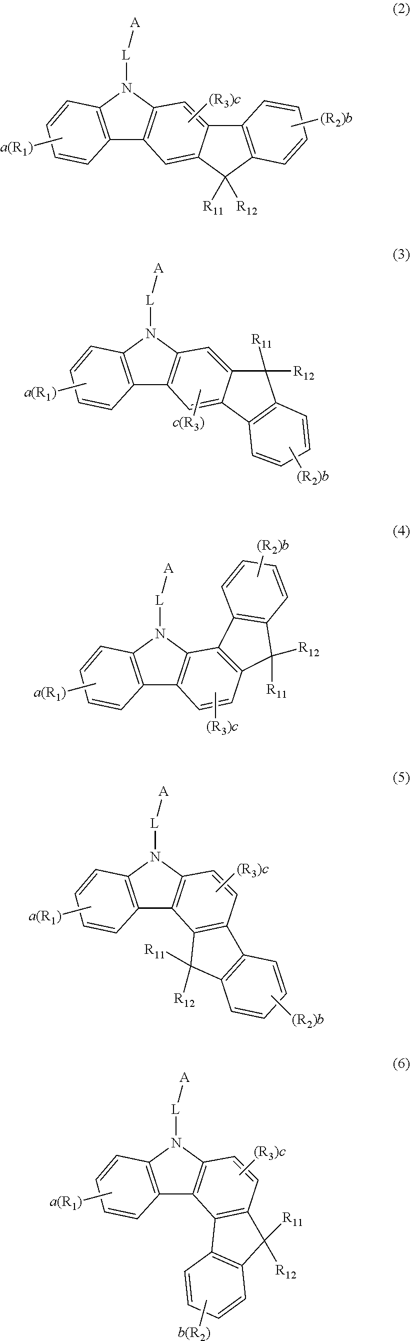

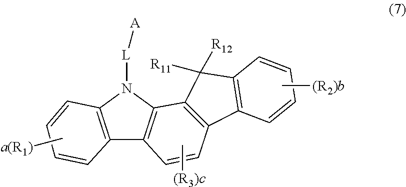

wherein A, L, R₁ to R₃, R₁₁, R₁₂, a, b, and c are as defined in formula 1.

Herein, “substituted” in the expression “substituted or unsubstituted” means that a hydrogen atom in a certain functional group is replaced with another atom or group, i.e., a substituent. The substituents of the substituted alkyl, the substituted alkoxy, the substituted cycloalkyl, the substituted aryl(ene), the substituted heteroaryl(ene), the substituted alkylsilyl, the substituted arylsilyl, the substituted arylalkylsilyl, the substituted arylamino, the substituted alkylamino, the substituted alkylarylamino, and the substituted arylalkyl in A, L, R₁ to R₃, R₁₁, and R₁₂ each independently are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 3- to 30-membered heteroaryl unsubstituted or substituted with a (C6-C30)aryl, a (C6-C30)aryl, a (C6-C30)aryl substituted with a 3- to 30-membered heteroaryl, a (C6-C30)aryl substituted with a tri(C1-C30)alkylsilyl, a (C6-C30)aryl substituted with a tri(C6-C30)arylsilyl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di- (C1-C30)alkylamino, a mono- or di- (C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl; and preferably each independently are at least one selected from the group consisting of a 5- to 20-membered heteroaryl unsubstituted or substituted with a (C6-C20)aryl, a (C6-C20)aryl, a (C6-C20)aryl substituted with a 5- to 20-membered heteroaryl, a (C6-C20)aryl substituted with a tri(C1-C6)alkylsilyl, a (C6-C20)aryl substituted with a tri(C6-C20)arylsilyl, and a (C1-C6)alkyl(C6-C20)aryl.

In formula 1, A represents a substituted or unsubstituted 5- to 30-membered heteroaryl; preferably represents a substituted or unsubstituted 5- to 20-membered heteroaryl; and more preferably represents an unsubstituted 5- to 20-membered heteroaryl, a 5- to 20-membered heteroaryl substituted with a 5- to 20-membered heteroaryl unsubstituted or substituted with a (C6-C20)aryl, a 5- to 20-membered heteroaryl substituted with a (C6-C20)aryl, a 5- to 20-membered heteroaryl substituted with a (C6-C20)aryl substituted with a 5- to 20-membered heteroaryl, a 5- to 20-membered heteroaryl substituted with a (C6-C20)aryl substituted with a tri(C1-C6)alkylsilyl, a 5- to 20-membered heteroaryl substituted with a (C6-C20)aryl substituted with a tri(C6-C20)arylsilyl, or a 5- to 20-membered heteroaryl substituted with a (C1-C6)alkyl(C6-C20)aryl.

In the definition of A, the 5- to 30-membered heteroaryl is preferably a nitrogen-containing heteroaryl, and more preferably a substituted or unsubstituted pyridine, a substituted or unsubstituted pyrimidine, a substituted or unsubstituted triazine, a substituted or unsubstituted pyrazine, a substituted or unsubstituted quinoline, a substituted or unsubstituted quinazoline, a substituted or unsubstituted quinoxaline, a substituted or unsubstituted naphthyridine, or a substituted or unsubstituted phenanthroline.

L represents a single bond, a substituted or unsubstituted (C6-C30)arylene, or a substituted or unsubstituted 5- to 30-membered heteroarylene; preferably represents a single bond, a substituted or unsubstituted (C6-C20)arylene, or a substituted or unsubstituted 5- to 20-membered heteroarylene; and more preferably represents a single bond, an unsubstituted (C6-C20)arylene, or an unsubstituted 5- to 20-membered heteroarylene.

X represents CR₁₁R₁₂.

R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring whose carbon atom(s) may be replaced with at least one hetero atom selected from nitrogen, oxygen, and sulfur; preferably each independently represent hydrogen, a substituted or unsubstituted (C6-C20)aryl, or a substituted or unsubstituted 5- to 20-membered heteroaryl; and more preferably each independently represent hydrogen, a (C6-C20)aryl unsubstituted or substituted with a (C6-C12)aryl, or a 5- to 20-membered heteroaryl unsubstituted or substituted with a (C6-C20)aryl.

R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring whose carbon atom(s) may be replaced with at least one hetero atom selected from nitrogen, oxygen, and sulfur; and preferably represents hydrogen.

R₁₁ and R₁₂ each independently represent a substituted or unsubstituted (C1-C30)alkyl, a substituted or unsubstituted (C6-C30)aryl, or a substituted or unsubstituted 5- to 30-membered heteroaryl; or are linked to each other to form a mono- or polycyclic (C3-C30) alicyclic or aromatic ring whose carbon atom(s) may be replaced with at least one hetero atom selected from nitrogen, oxygen, and sulfur; preferably each independently represent a substituted or unsubstituted (C1-C6)alkyl, or a substituted or unsubstituted (C6-C20)aryl; or are linked to each other to form a mono- or polycyclic (C5-C20) alicyclic or aromatic ring; and more preferably each independently represent an unsubstituted (C1-C6)alkyl, or an unsubstituted (C6-C20)aryl; or are linked to each other to form a mono- or polycyclic (C5-C20) alicyclic or aromatic ring.

a and b each independently represent an integer of 1 to 4, preferably an integer of 1 to 2, where a or b is an integer of 2 or more, each of R₁ and each of R₂ may be the same or different.

c represents an integer of 1 to 2, and preferably 1.

The heteroaryl(ene) contains at least one hetero atom selected from B, N, O, S, P(═O), Si, and P.

According to one embodiment of the present invention, in formula 1, A represents a substituted or unsubstituted 5- to 20-membered heteroaryl; L represents a single bond, a substituted or unsubstituted (C6-C20)arylene, or a substituted or unsubstituted 5- to 20-membered heteroarylene; X represents CR₁₁R₁₂; R₁ and R₂ each independently represent hydrogen, a substituted or unsubstituted (C6-C20)aryl, or a substituted or unsubstituted 5- to 20-membered heteroaryl; R₃ represents hydrogen; R₁₁ and R₁₂ each independently represent a substituted or unsubstituted (C1-C6)alkyl, or a substituted or unsubstituted (C6-C20)aryl, or are linked to each other to form a mono- or polycyclic (C5-C20) alicyclic or aromatic ring; a and b each independently represent an integer of 1 to 2; and c represents 1.

According to another embodiment of the present invention, in formula 1, A represents an unsubstituted 5- to 20-membered heteroaryl, a 5- to 20-membered heteroaryl substituted with a 5- to 20-membered heteroaryl unsubstituted or substituted with a (C6-C20)aryl, a 5- to 20-membered heteroaryl substituted with a (C6-C20)aryl, a 5- to 20-membered heteroaryl substituted with a (C6-C20)aryl substituted with a 5- to 20-membered heteroaryl, a 5- to 20-membered heteroaryl substituted with a (C6-C20)aryl substituted with a tri(C1-C6)alkylsilyl, a 5- to 20-membered heteroaryl substituted with a (C6-C20)aryl substituted with a tri(C6-C20)arylsilyl, or a 5- to 20-membered heteroaryl substituted with a (C1-C6)alkyl(C6-C20)aryl; L represents a single bond, an unsubstituted (C6-C20)arylene, or an unsubstituted 5- to 20-membered heteroarylene; X represents CR₁₁R₁₂; R₁ and R₂ each independently represent hydrogen, a (C6-C20)aryl unsubstituted or substituted with a (C6-C12)aryl, or a 5- to 20-membered heteroaryl unsubstituted or substituted with a (C6-C20)aryl; R₃ represents hydrogen; R₁₁ and R₁₂ each independently represent an unsubstituted (C1-C6)alkyl, or an unsubstituted (C6-C20)aryl, or are linked to each other to form a mono- or polycyclic (C5-C20) alicyclic or aromatic ring; a and b each independently represent an integer of 1 to 2; and c represents 1.

The compound of formula 1 may be selected from the group consisting of the following compounds, but is not limited thereto:

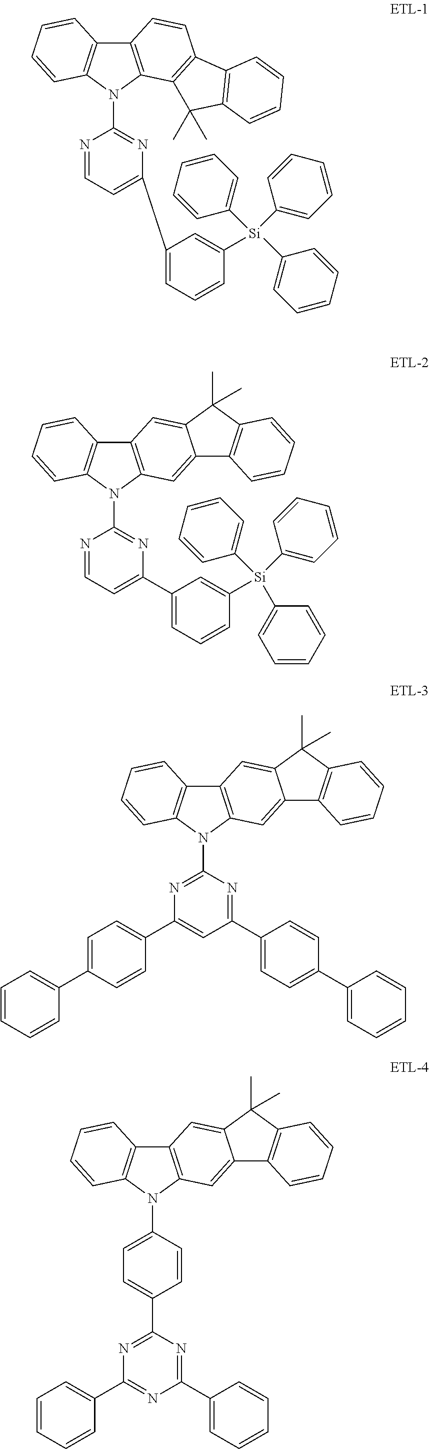 [SMILES: CC1(C)c2ccccc2-c2ccc3c4ccccc4n(-c4nccc(-c5cccc([Si](c6ccccc6)(c6ccccc6)c6ccccc6)c5)n4)c3c21 ; CC1(C)c2ccccc2-c2cc3c(cc21)c1ccccc1n3-c1nccc(-c2cccc([Si](c3ccccc3)(c3ccccc3)c3ccccc3)c2)n1 ; CC1(C)c2ccccc2-c2cc3c(cc21)c1ccccc1n3-c1nc(-c2ccc(-c3ccccc3)cc2)cc(-c2ccc(-c3ccccc3)cc2)n1 ; CC1(C)c2ccccc2-c2cc3c(cc21)c1ccccc1n3-c1ccc(-c2nc(-c3ccccc3)nc(-c3ccccc3)n2)cc1]

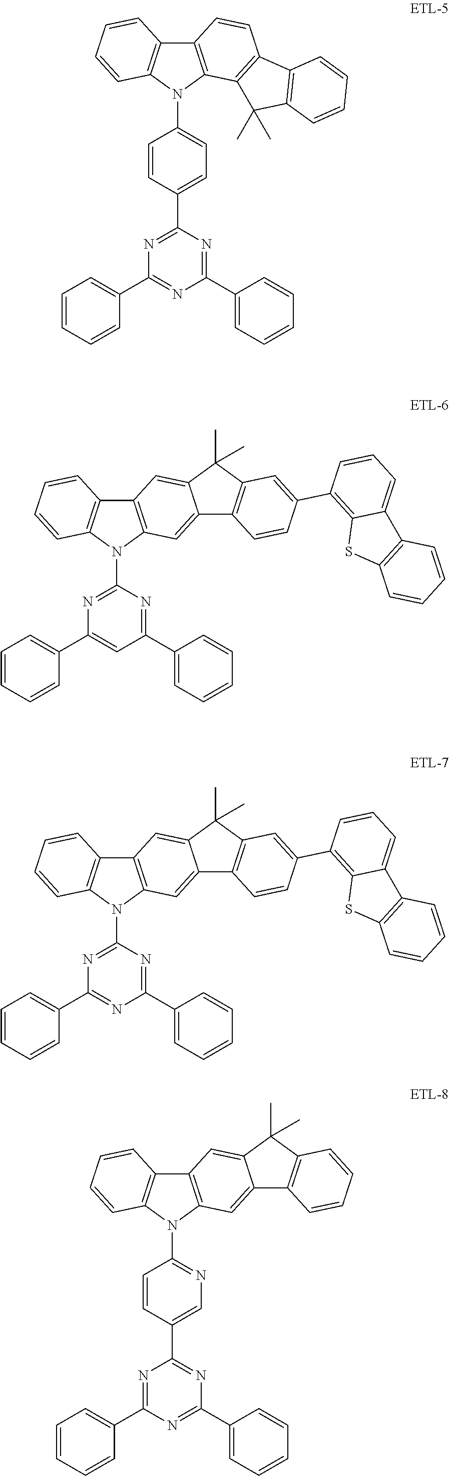 [SMILES: CC1(C)c2ccccc2-c2ccc3c4ccccc4n(-c4ccc(-c5nc(-c6ccccc6)nc(-c6ccccc6)n5)cc4)c3c21 ; CC1(C)c2cc(-c3cccc4c3sc3ccccc34)ccc2-c2cc3c(cc21)c1ccccc1n3-c1nc(-c2ccccc2)cc(-c2ccccc2)n1 ; CC1(C)c2cc(-c3cccc4c3sc3ccccc34)ccc2-c2cc3c(cc21)c1ccccc1n3-c1nc(-c2ccccc2)nc(-c2ccccc2)n1 ; CC1(C)c2ccccc2-c2cc3c(cc21)c1ccccc1n3-c1ccc(-c2nc(-c3ccccc3)nc(-c3ccccc3)n2)cn1]

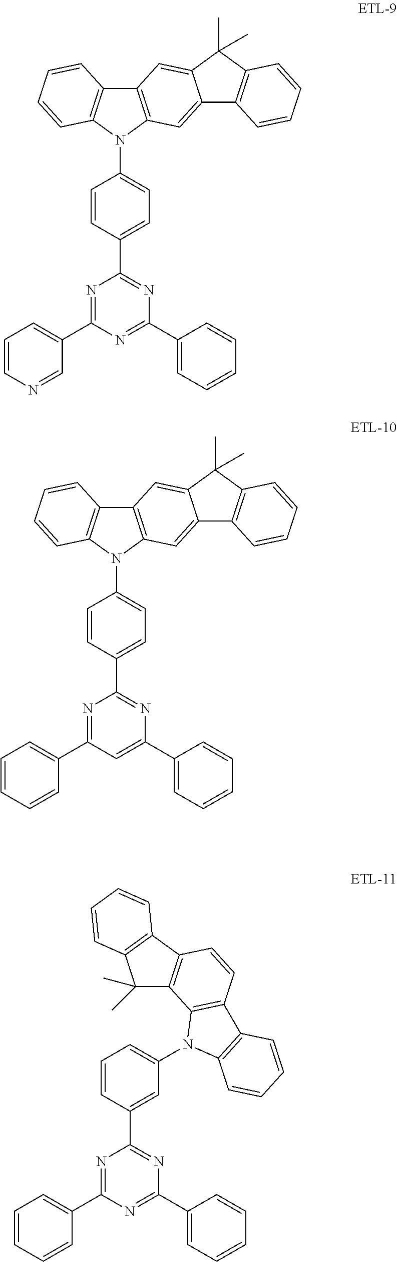 [SMILES: CC1(C)c2ccccc2-c2cc3c(cc21)c1ccccc1n3-c1ccc(-c2nc(-c3ccccc3)nc(-c3cccnc3)n2)cc1 ; CC1(C)c2ccccc2-c2cc3c(cc21)c1ccccc1n3-c1ccc(-c2nc(-c3ccccc3)cc(-c3ccccc3)n2)cc1 ; CC1(C)c2ccccc2-c2ccc3c4ccccc4n(-c4cccc(-c5nc(-c6ccccc6)nc(-c6ccccc6)n5)c4)c3c21]

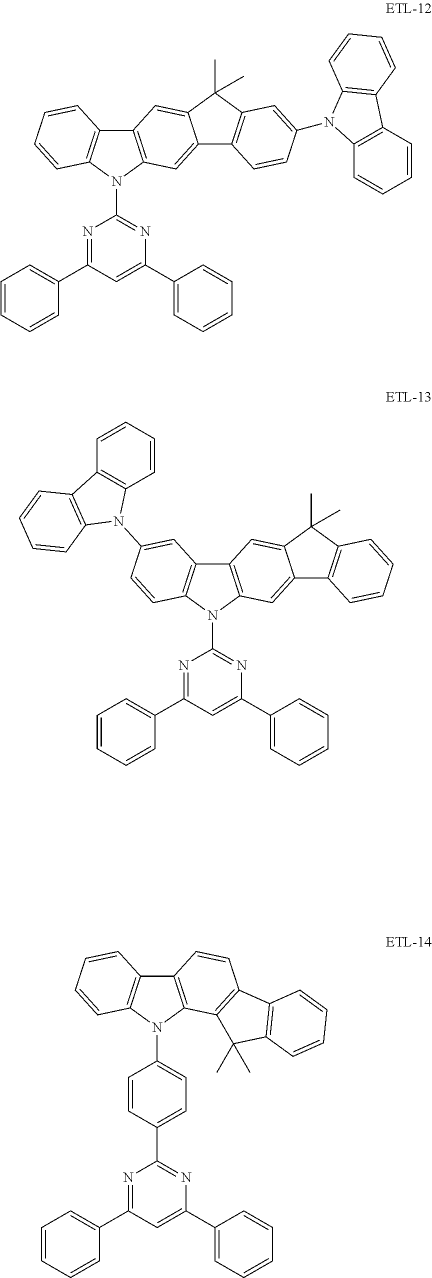 [SMILES: CC1(C)c2cc(-n3c4ccccc4c4ccccc43)ccc2-c2cc3c(cc21)c1ccccc1n3-c1nc(-c2ccccc2)cc(-c2ccccc2)n1 ; CC1(C)c2ccccc2-c2cc3c(cc21)c1cc(-n2c4ccccc4c4ccccc42)ccc1n3-c1nc(-c2ccccc2)cc(-c2ccccc2)n1 ; CC1(C)c2ccccc2-c2ccc3c4ccccc4n(-c4ccc(-c5nc(-c6ccccc6)cc(-c6ccccc6)n5)cc4)c3c21]

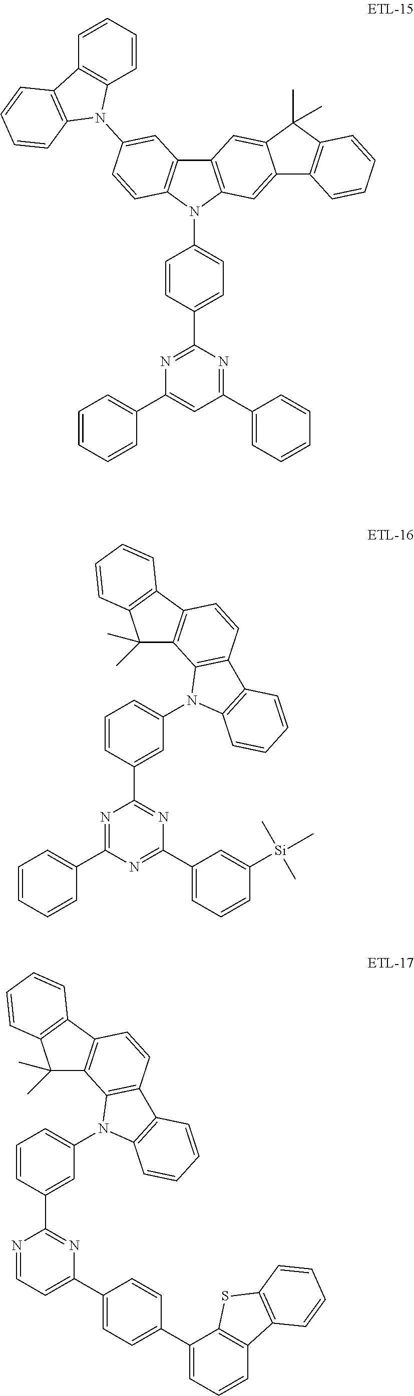 [SMILES: CC1(C)c2ccccc2-c2cc3c(cc21)c1cc(-n2c4ccccc4c4ccccc42)ccc1n3-c1ccc(-c2nc(-c3ccccc3)cc(-c3ccccc3)n2)cc1 ; CC1(C)c2ccccc2-c2ccc3c4ccccc4n(-c4cccc(-c5nc(-c6ccccc6)nc(-c6cccc([Si](C)(C)C)c6)n5)c4)c3c21 ; CC1(C)c2ccccc2-c2ccc3c4ccccc4n(-c4cccc(-c5nccc(-c6ccc(-c7cccc8c7sc7ccccc78)cc6)n5)c4)c3c21]

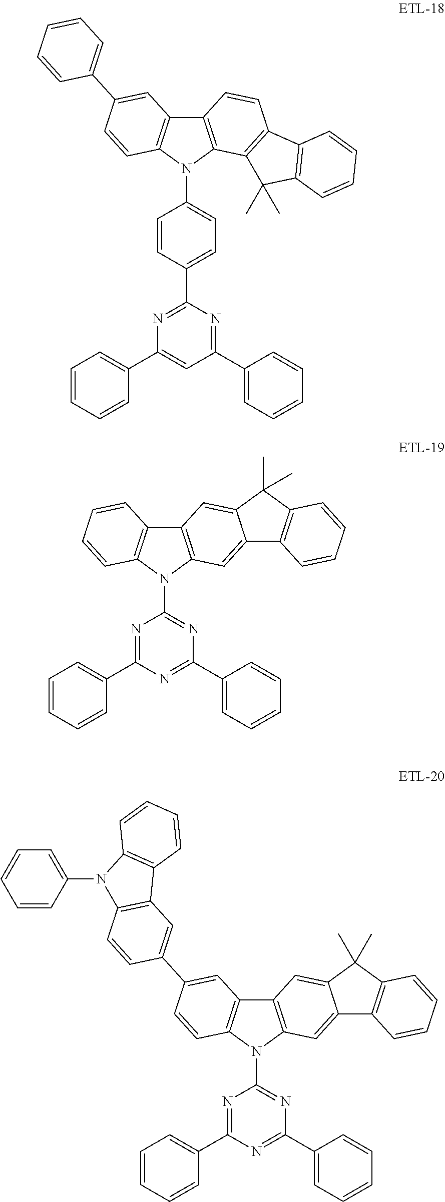

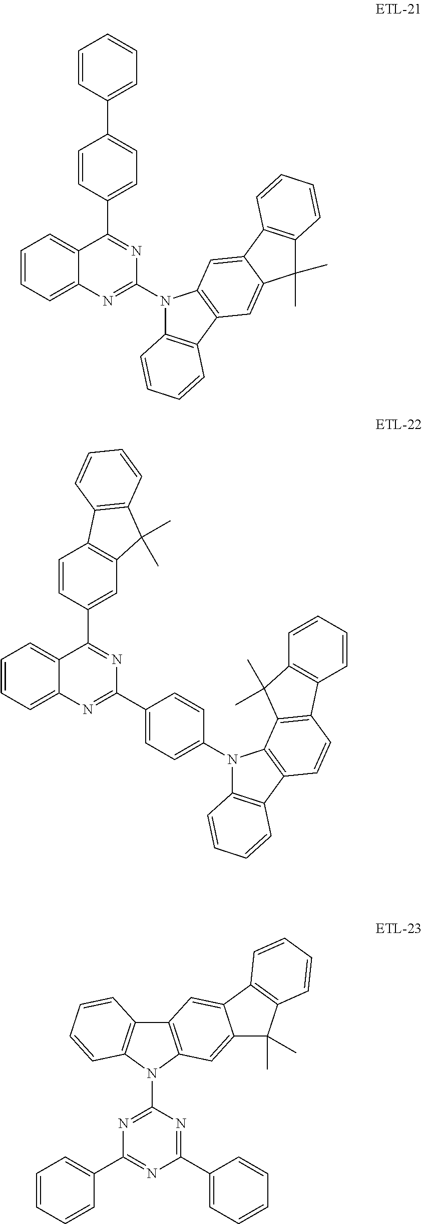 [SMILES: CC1(C)c2ccccc2-c2cc3c(cc21)c1ccccc1n3-c1nc(-c2ccc(-c3ccccc3)cc2)c2ccccc2n1 ; CC1(C)c2ccccc2-c2ccc(-c3nc(-c4ccc(-n5c6ccccc6c6ccc7c(c65)C(C)(C)c5ccccc5-7)cc4)nc4ccccc34)cc21 ; CC1(C)c2ccccc2-c2cc3c4ccccc4n(-c4nc(-c5ccccc5)nc(-c5ccccc5)n4)c3cc21]

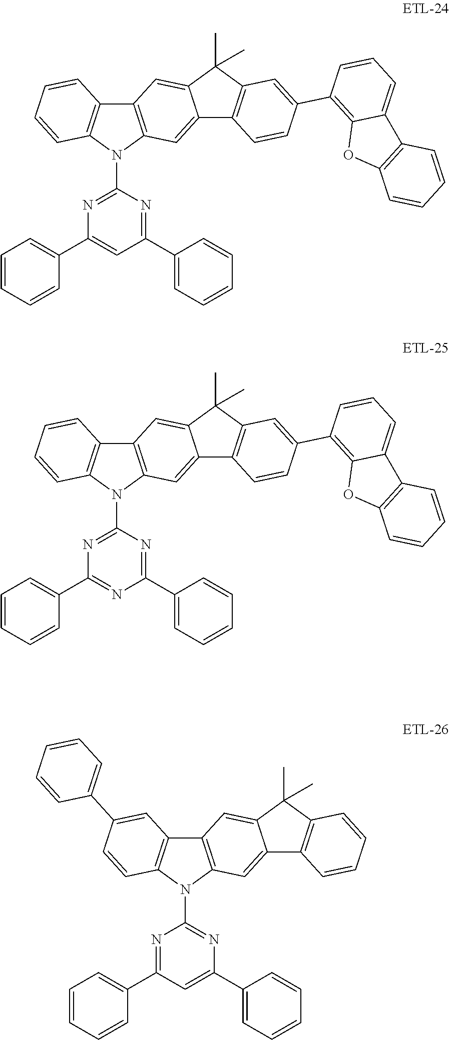 [SMILES: CC1(C)c2cc(-c3cccc4c3oc3ccccc34)ccc2-c2cc3c(cc21)c1ccccc1n3-c1nc(-c2ccccc2)cc(-c2ccccc2)n1 ; CC1(C)c2cc(-c3cccc4c3oc3ccccc34)ccc2-c2cc3c(cc21)c1ccccc1n3-c1nc(-c2ccccc2)nc(-c2ccccc2)n1 ; CC1(C)c2ccccc2-c2cc3c(cc21)c1cc(-c2ccccc2)ccc1n3-c1nc(-c2ccccc2)cc(-c2ccccc2)n1]

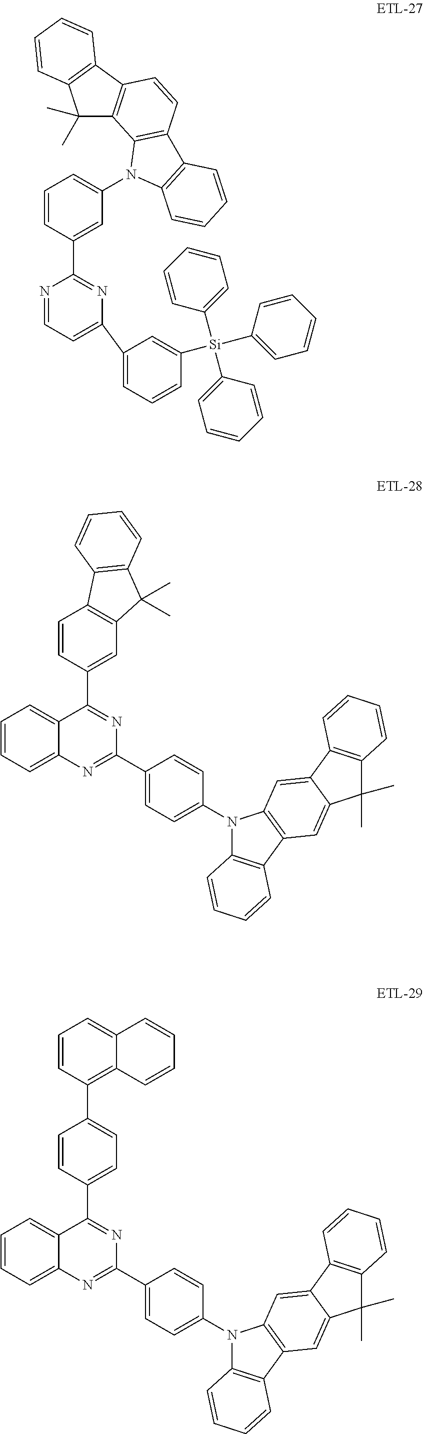 [SMILES: CC1(C)c2ccccc2-c2ccc3c4ccccc4n(-c4cccc(-c5nccc(-c6cccc([Si](c7ccccc7)(c7ccccc7)c7ccccc7)c6)n5)c4)c3c21 ; CC1(C)c2ccccc2-c2ccc(-c3nc(-c4ccc(-n5c6ccccc6c6cc7c(cc65)-c5ccccc5C7(C)C)cc4)nc4ccccc34)cc21 ; CC1(C)c2ccccc2-c2cc3c(cc21)c1ccccc1n3-c1ccc(-c2nc(-c3ccc(-c4cccc5ccccc45)cc3)c3ccccc3n2)cc1]

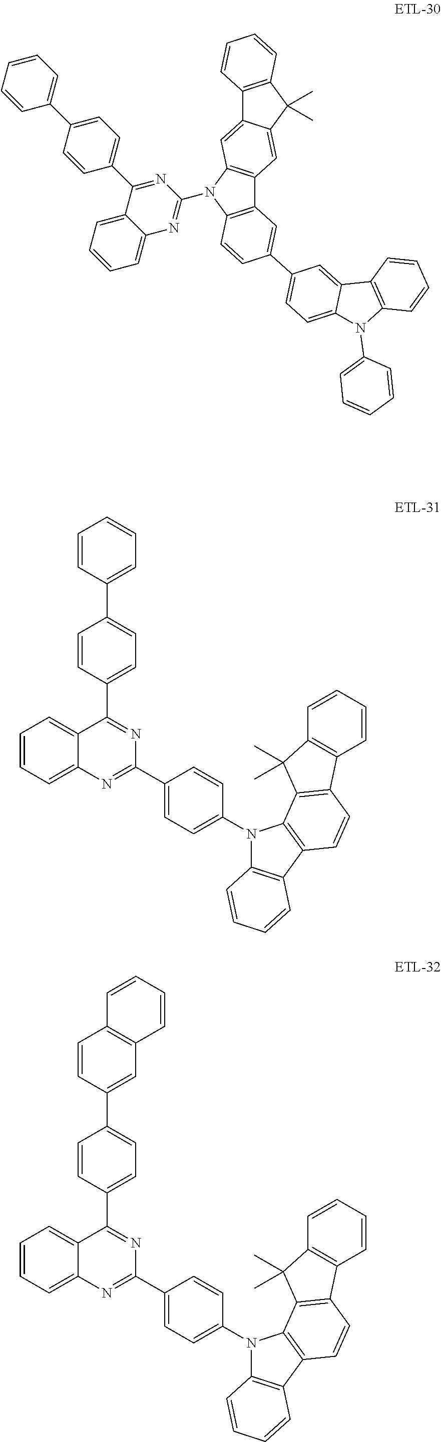 [SMILES: CC1(C)c2ccccc2-c2cc3c(cc21)c1cc(-c2ccc4c(c2)c2ccccc2n4-c2ccccc2)ccc1n3-c1nc(-c2ccc(-c3ccccc3)cc2)c2ccccc2n1 ; CC1(C)c2ccccc2-c2ccc3c4ccccc4n(-c4ccc(-c5nc(-c6ccc(-c7ccccc7)cc6)c6ccccc6n5)cc4)c3c21 ; CC1(C)c2ccccc2-c2ccc3c4ccccc4n(-c4ccc(-c5nc(-c6ccc(-c7ccc8ccccc8c7)cc6)c6ccccc6n5)cc4)c3c21]

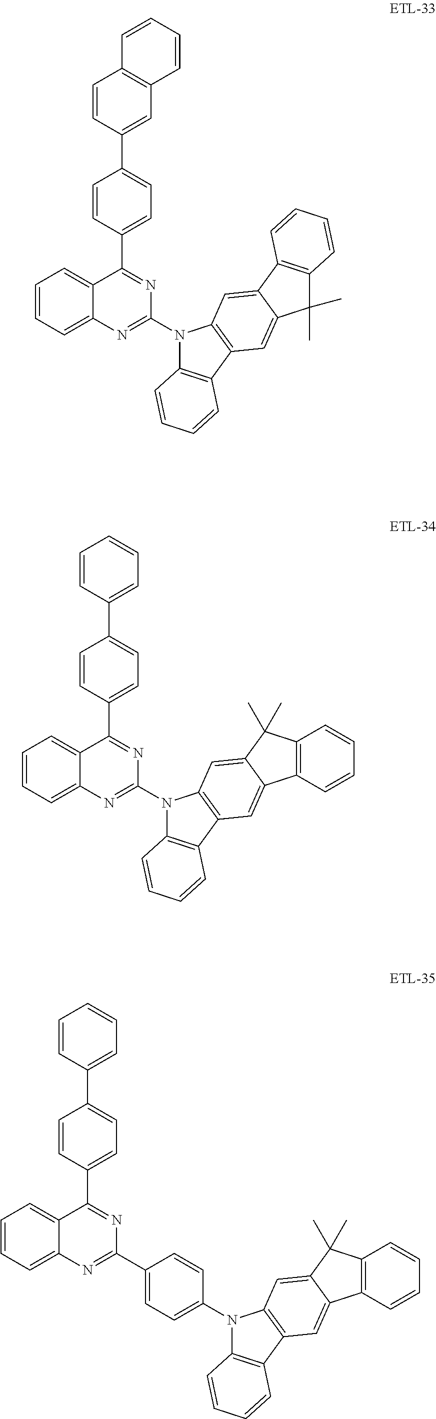 [SMILES: CC1(C)c2ccccc2-c2cc3c(cc21)c1ccccc1n3-c1nc(-c2ccc(-c3ccc4ccccc4c3)cc2)c2ccccc2n1 ; CC1(C)c2ccccc2-c2cc3c4ccccc4n(-c4nc(-c5ccc(-c6ccccc6)cc5)c5ccccc5n4)c3cc21 ; CC1(C)c2ccccc2-c2cc3c4ccccc4n(-c4ccc(-c5nc(-c6ccc(-c7ccccc7)cc6)c6ccccc6n5)cc4)c3cc21]

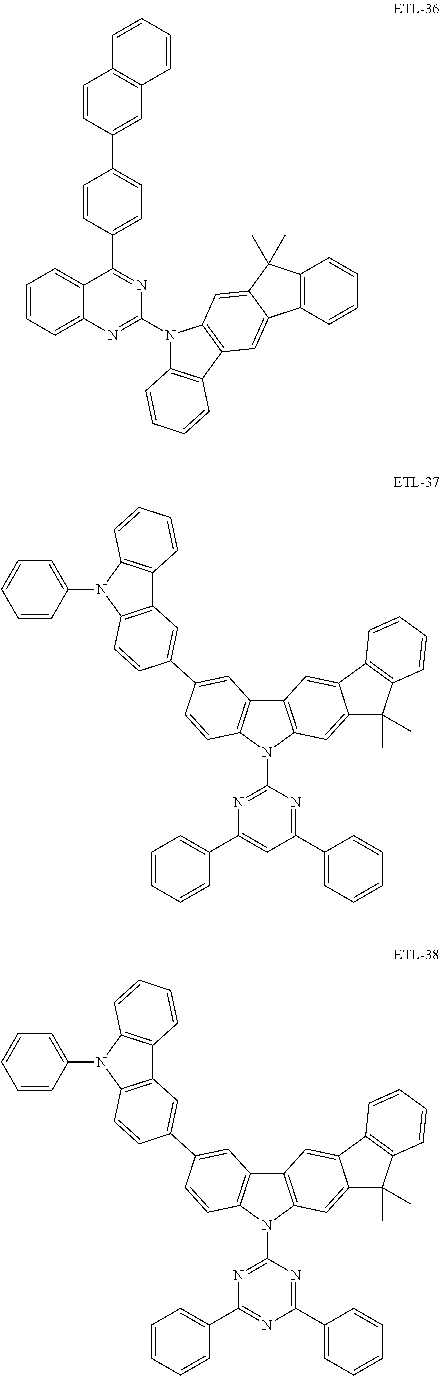 [SMILES: CC1(C)c2ccccc2-c2cc3c4ccccc4n(-c4nc(-c5ccc(-c6ccc7ccccc7c6)cc5)c5ccccc5n4)c3cc21 ; CC1(C)c2ccccc2-c2cc3c4cc(-c5ccc6c(c5)c5ccccc5n6-c5ccccc5)ccc4n(-c4nc(-c5ccccc5)cc(-c5ccccc5)n4)c3cc21 ; CC1(C)c2ccccc2-c2cc3c4cc(-c5ccc6c(c5)c5ccccc5n6-c5ccccc5)ccc4n(-c4nc(-c5ccccc5)nc(-c5ccccc5)n4)c3cc21]

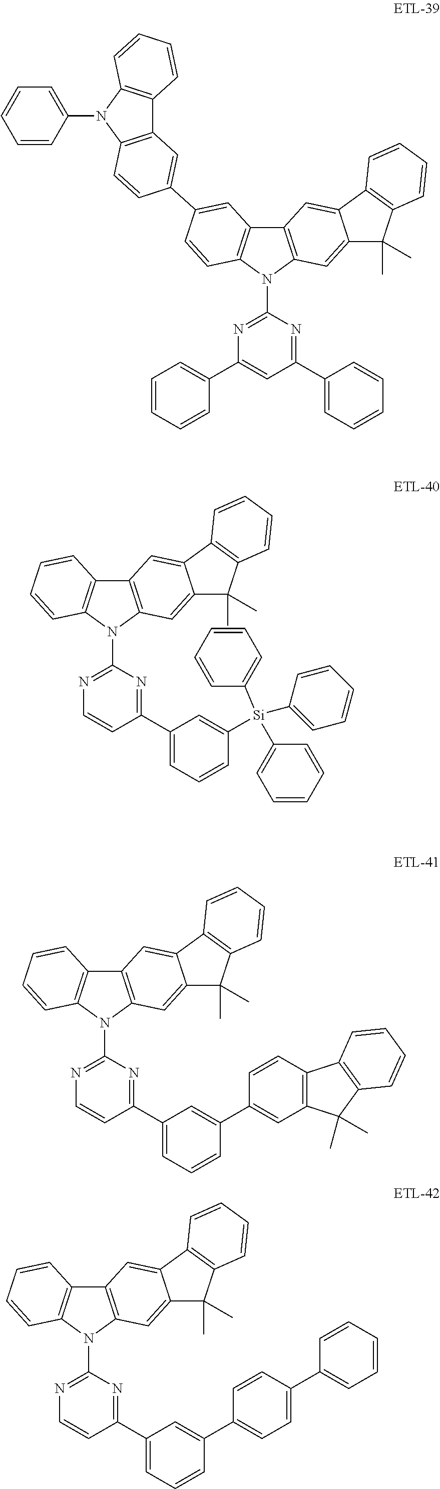

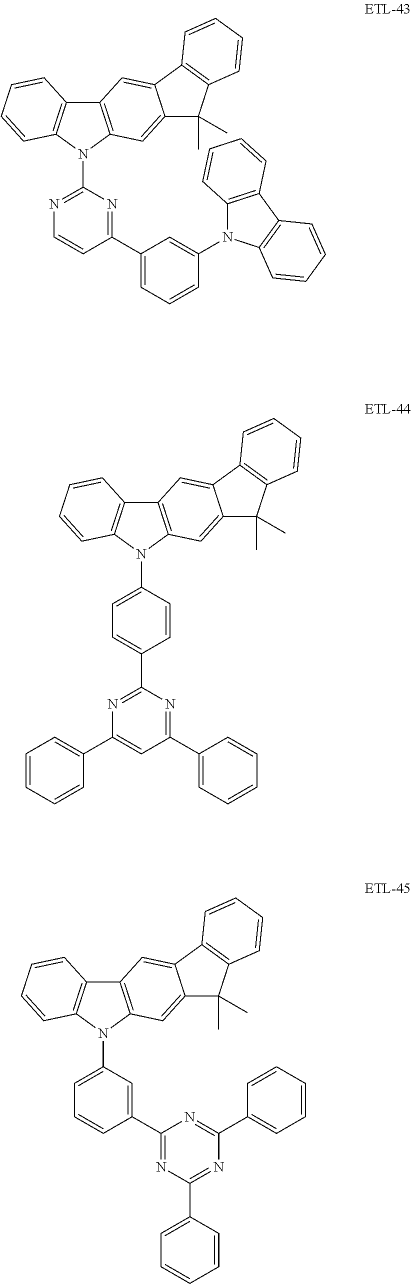 [SMILES: CC1(C)c2ccccc2-c2cc3c4ccccc4n(-c4nccc(-c5cccc(-n6c7ccccc7c7ccccc76)c5)n4)c3cc21 ; CC1(C)c2ccccc2-c2cc3c4ccccc4n(-c4ccc(-c5nc(-c6ccccc6)cc(-c6ccccc6)n5)cc4)c3cc21 ; CC1(C)c2ccccc2-c2cc3c4ccccc4n(-c4cccc(-c5nc(-c6ccccc6)nc(-c6ccccc6)n5)c4)c3cc21]

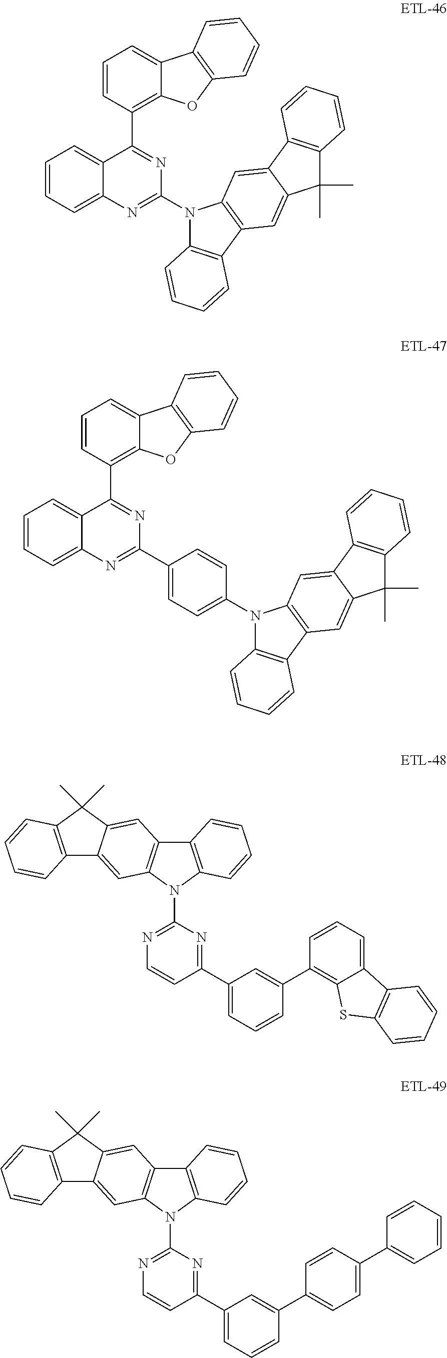 [SMILES: CC1(C)c2ccccc2-c2cc3c(cc21)c1ccccc1n3-c1nc(-c2cccc3c2oc2ccccc23)c2ccccc2n1 ; CC1(C)c2ccccc2-c2cc3c(cc21)c1ccccc1n3-c1ccc(-c2nc(-c3cccc4c3oc3ccccc34)c3ccccc3n2)cc1 ; CC1(C)c2ccccc2-c2cc3c(cc21)c1ccccc1n3-c1nccc(-c2cccc(-c3cccc4c3sc3ccccc34)c2)n1 ; CC1(C)c2ccccc2-c2cc3c(cc21)c1ccccc1n3-c1nccc(-c2cccc(-c3ccc(-c4ccccc4)cc3)c2)n1]

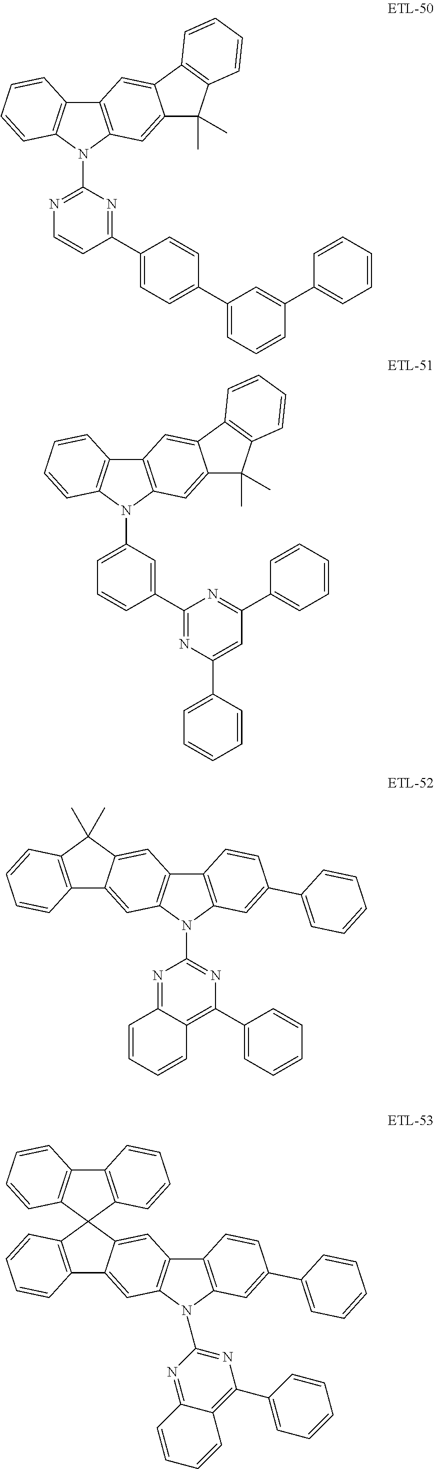

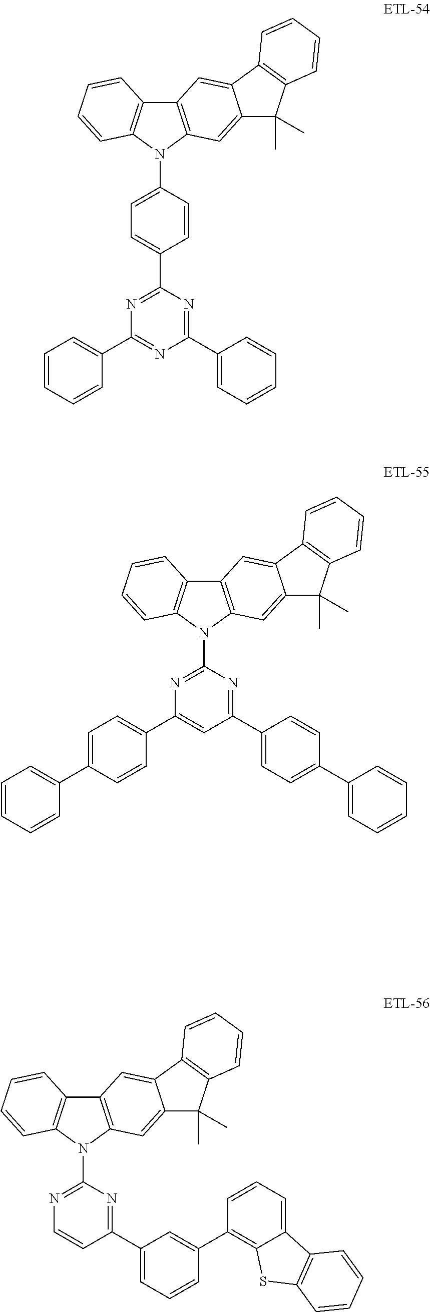 [SMILES: CC1(C)c2ccccc2-c2cc3c4ccccc4n(-c4ccc(-c5nc(-c6ccccc6)nc(-c6ccccc6)n5)cc4)c3cc21 ; CC1(C)c2ccccc2-c2cc3c4ccccc4n(-c4nc(-c5ccc(-c6ccccc6)cc5)cc(-c5ccc(-c6ccccc6)cc5)n4)c3cc21 ; CC1(C)c2ccccc2-c2cc3c4ccccc4n(-c4nccc(-c5cccc(-c6cccc7c6sc6ccccc67)c5)n4)c3cc21]

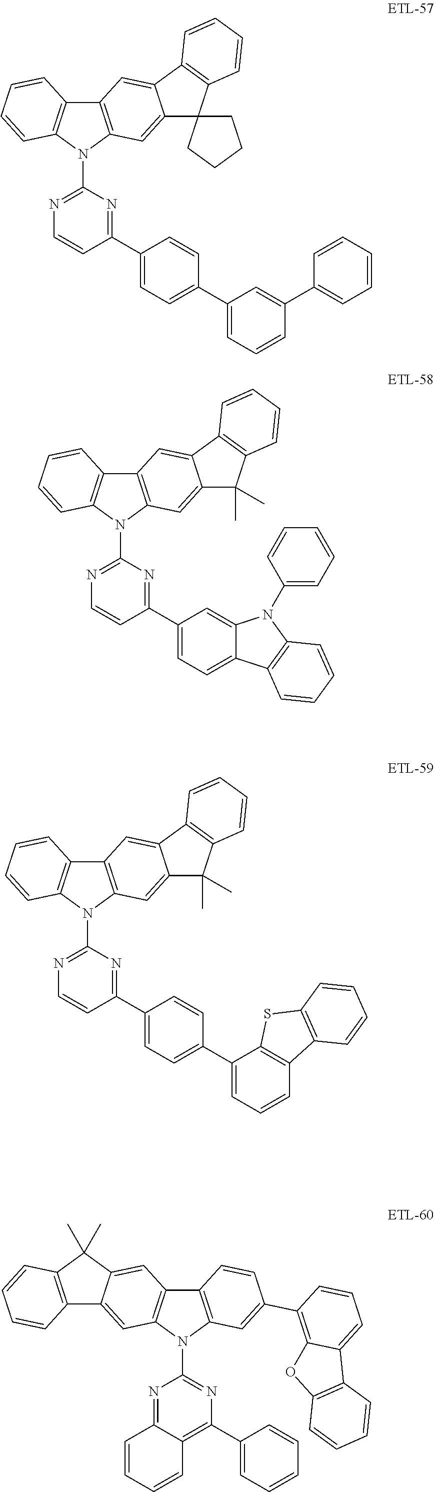

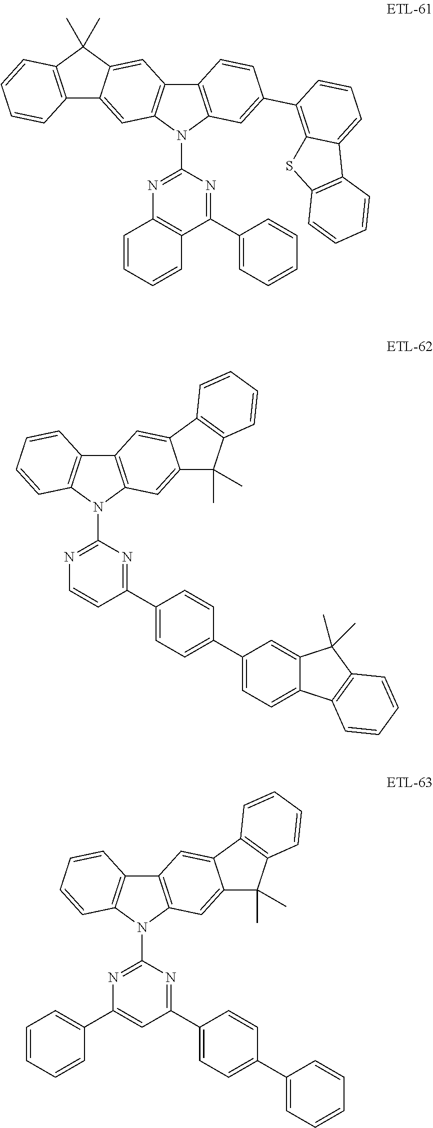 [SMILES: CC1(C)c2ccccc2-c2cc3c(cc21)c1ccc(-c2cccc4c2sc2ccccc24)cc1n3-c1nc(-c2ccccc2)c2ccccc2n1 ; CC1(C)c2ccccc2-c2ccc(-c3ccc(-c4ccnc(-n5c6ccccc6c6cc7c(cc65)C(C)(C)c5ccccc5-7)n4)cc3)cc21 ; CC1(C)c2ccccc2-c2cc3c4ccccc4n(-c4nc(-c5ccccc5)cc(-c5ccc(-c6ccccc6)cc5)n4)c3cc21]

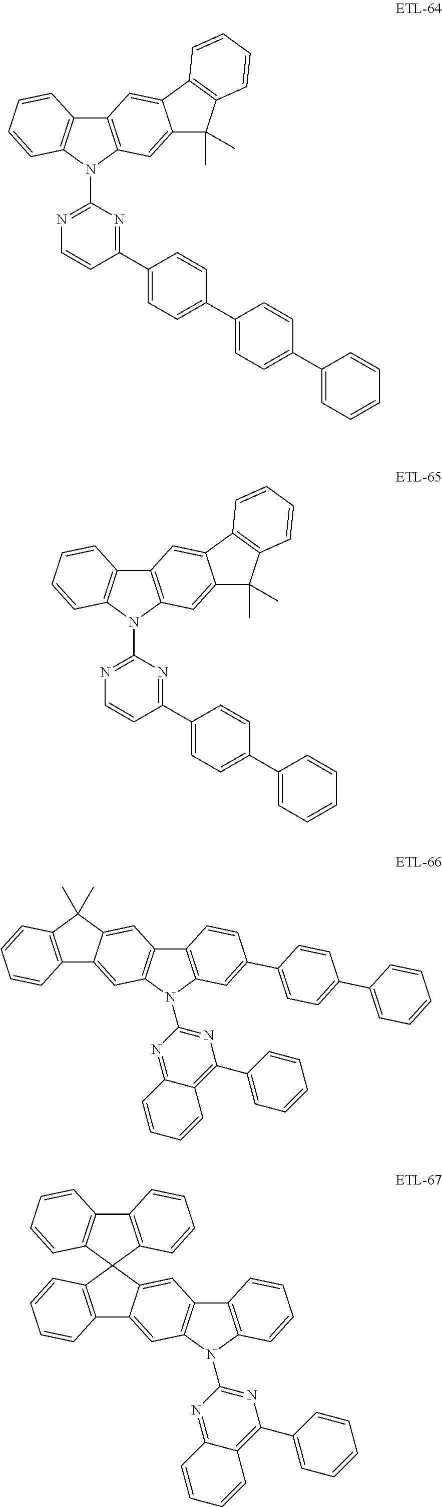 [SMILES: CC1(C)c2ccccc2-c2cc3c4ccccc4n(-c4nccc(-c5ccc(-c6ccc(-c7ccccc7)cc6)cc5)n4)c3cc21 ; CC1(C)c2ccccc2-c2cc3c4ccccc4n(-c4nccc(-c5ccc(-c6ccccc6)cc5)n4)c3cc21 ; CC1(C)c2ccccc2-c2cc3c(cc21)c1ccc(-c2ccc(-c4ccccc4)cc2)cc1n3-c1nc(-c2ccccc2)c2ccccc2n1 ; c1ccc(-c2nc(-n3c4ccccc4c4cc5c(cc43)-c3ccccc3C53c4ccccc4-c4ccccc43)nc3ccccc23)cc1]

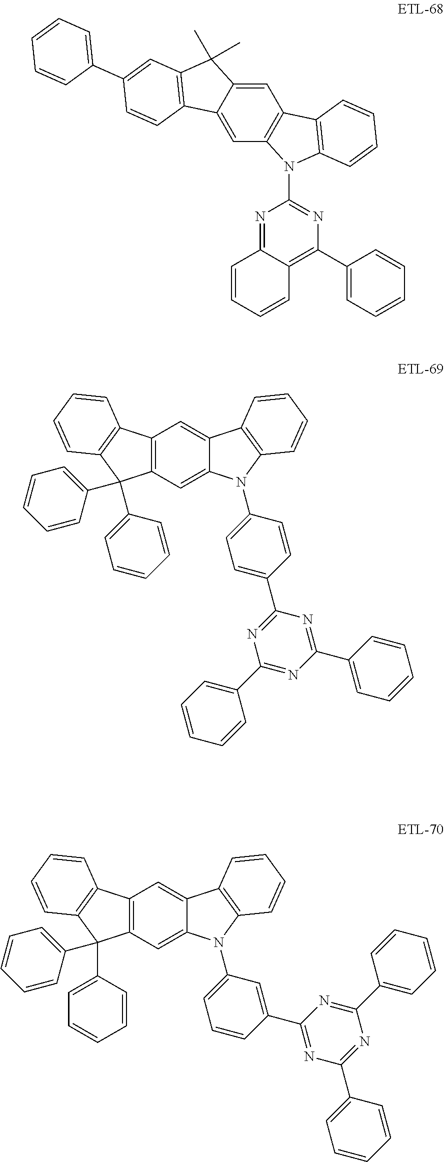 [SMILES: CC1(C)c2cc(-c3ccccc3)ccc2-c2cc3c(cc21)c1ccccc1n3-c1nc(-c2ccccc2)c2ccccc2n1 ; c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1 ; c1ccc(-c2nc(-c3ccccc3)nc(-c3cccc(-n4c5ccccc5c5cc6c(cc54)C(c4ccccc4)(c4ccccc4)c4ccccc4-6)c3)n2)cc1]

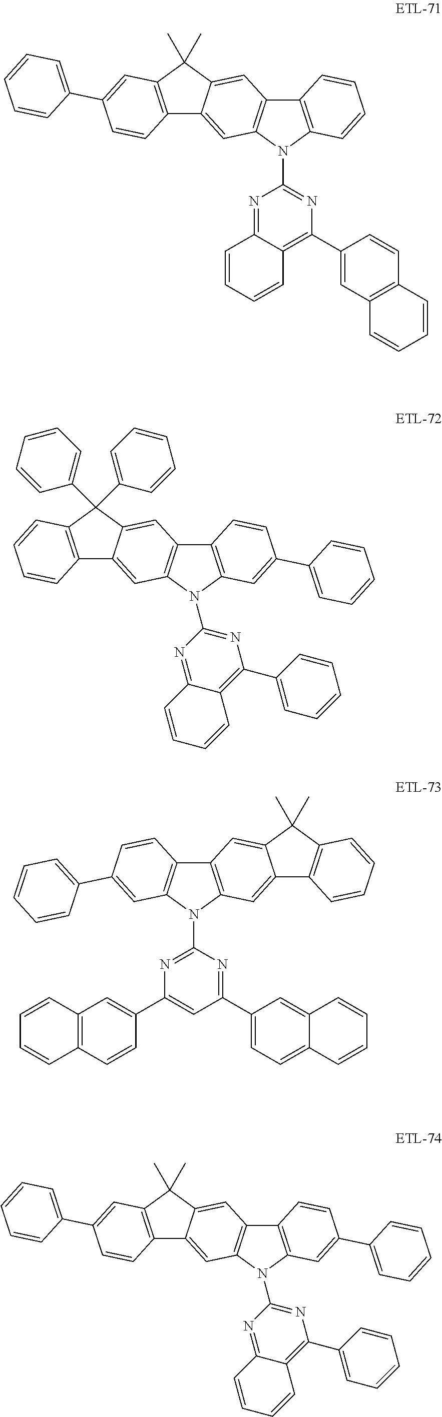 [SMILES: CC1(C)c2cc(-c3ccccc3)ccc2-c2cc3c(cc21)c1ccccc1n3-c1nc(-c2ccc3ccccc3c2)c2ccccc2n1 ; c1ccc(-c2ccc3c4cc5c(cc4n(-c4nc(-c6ccccc6)c6ccccc6n4)c3c2)-c2ccccc2C5(c2ccccc2)c2ccccc2)cc1 ; CC1(C)c2ccccc2-c2cc3c(cc21)c1ccc(-c2ccccc2)cc1n3-c1nc(-c2ccc3ccccc3c2)cc(-c2ccc3ccccc3c2)n1 ; CC1(C)c2cc(-c3ccccc3)ccc2-c2cc3c(cc21)c1ccc(-c2ccccc2)cc1n3-c1nc(-c2ccccc2)c2ccccc2n1]

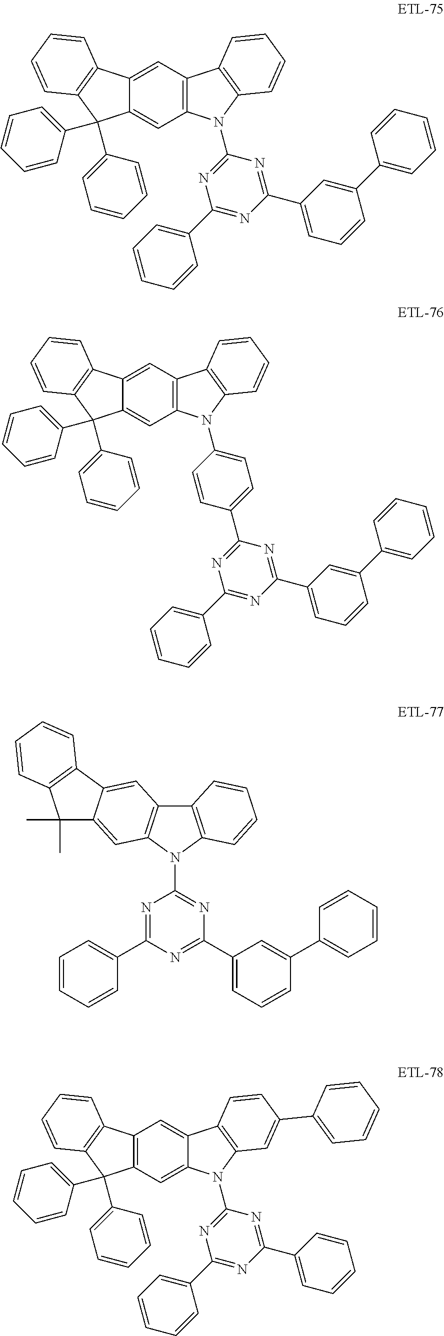 [SMILES: c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; c1ccc(-c2cccc(-c3nc(-c4ccccc4)nc(-c4ccc(-n5c6ccccc6c6cc7c(cc65)C(c5ccccc5)(c5ccccc5)c5ccccc5-7)cc4)n3)c2)cc1 ; CC1(C)c2ccccc2-c2cc3c4ccccc4n(-c4nc(-c5ccccc5)nc(-c5cccc(-c6ccccc6)c5)n4)c3cc21 ; c1ccc(-c2ccc3c4cc5c(cc4n(-c4nc(-c6ccccc6)nc(-c6ccccc6)n4)c3c2)C(c2ccccc2)(c2ccccc2)c2ccccc2-5)cc1]

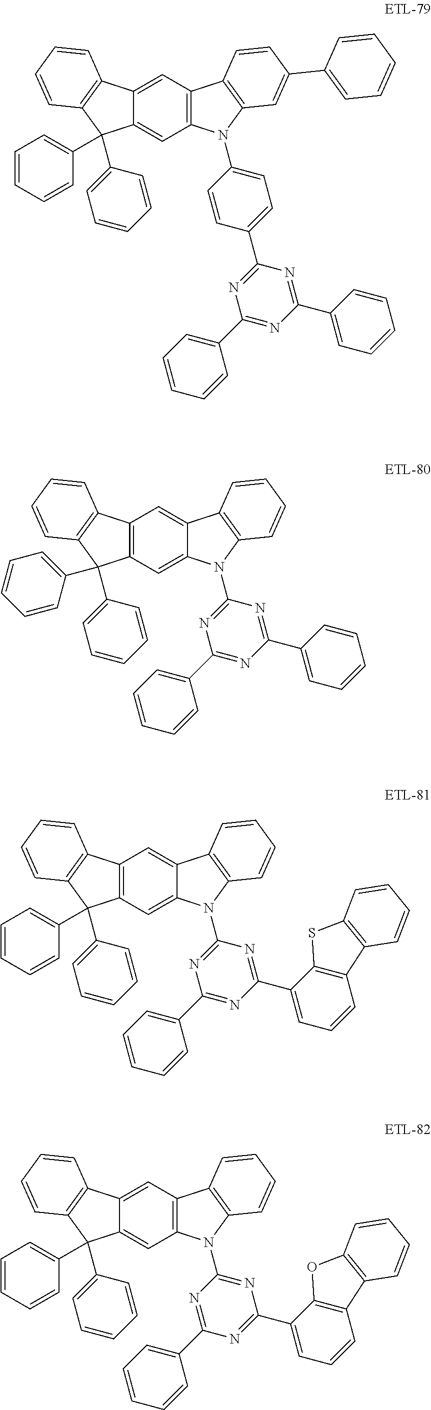 [SMILES: c1ccc(-c2ccc3c4cc5c(cc4n(-c4ccc(-c6nc(-c7ccccc7)nc(-c7ccccc7)n6)cc4)c3c2)C(c2ccccc2)(c2ccccc2)c2ccccc2-5)cc1 ; c1ccc(-c2nc(-c3ccccc3)nc(-n3c4ccccc4c4cc5c(cc43)C(c3ccccc3)(c3ccccc3)c3ccccc3-5)n2)cc1 ; c1ccc(-c2nc(-c3cccc4c3sc3ccccc34)nc(-n3c4ccccc4c4cc5c(cc43)C(c3ccccc3)(c3ccccc3)c3ccccc3-5)n2)cc1 ; c1ccc(-c2nc(-c3cccc4c3oc3ccccc34)nc(-n3c4ccccc4c4cc5c(cc43)C(c3ccccc3)(c3ccccc3)c3ccccc3-5)n2)cc1]

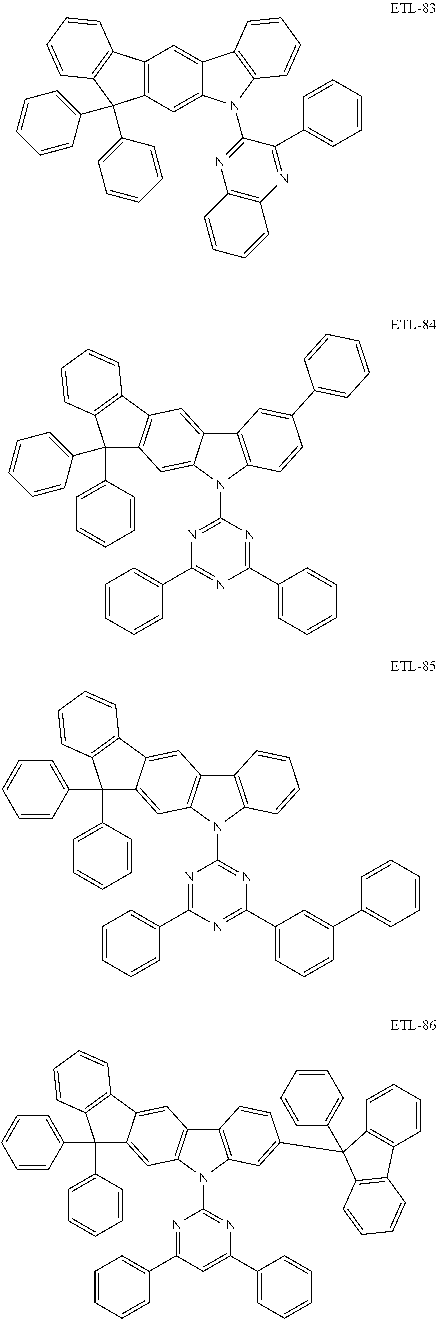 [SMILES: c1ccc(-c2nc3ccccc3nc2-n2c3ccccc3c3cc4c(cc32)C(c2ccccc2)(c2ccccc2)c2ccccc2-4)cc1 ; c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1 ; c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; c1ccc(-c2cc(-c3ccccc3)nc(-n3c4cc(C5(c6ccccc6)c6ccccc6-c6ccccc65)ccc4c4cc5c(cc43)C(c3ccccc3)(c3ccccc3)c3ccccc3-5)n2)cc1]

The compound of formula 1 comprised in the electron transport material according to the present invention can be prepared by known methods to one skilled in the art, and can be prepared, for example, according to the following reaction scheme:

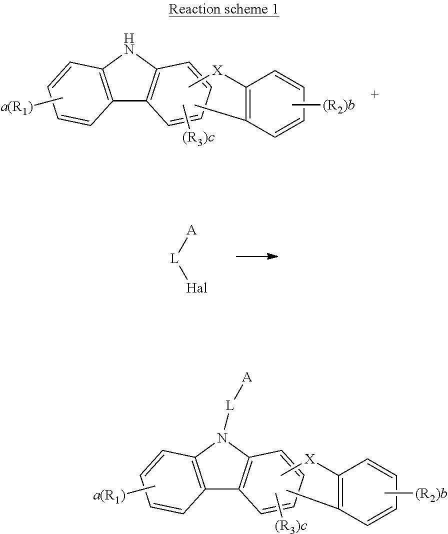

wherein

A, L, X, R₁ to R₃, a, b, and c are as defined in formula 1, and Hal represents a halogen.

The present invention provides an electron transport material comprising the compound of formula 1, and an organic EL device comprising the material. The electron transport material can be comprised of the compound of formula 1 alone, or can be a mixture or composition for an electron transport layer which further comprises conventional materials generally included in electron transport materials.

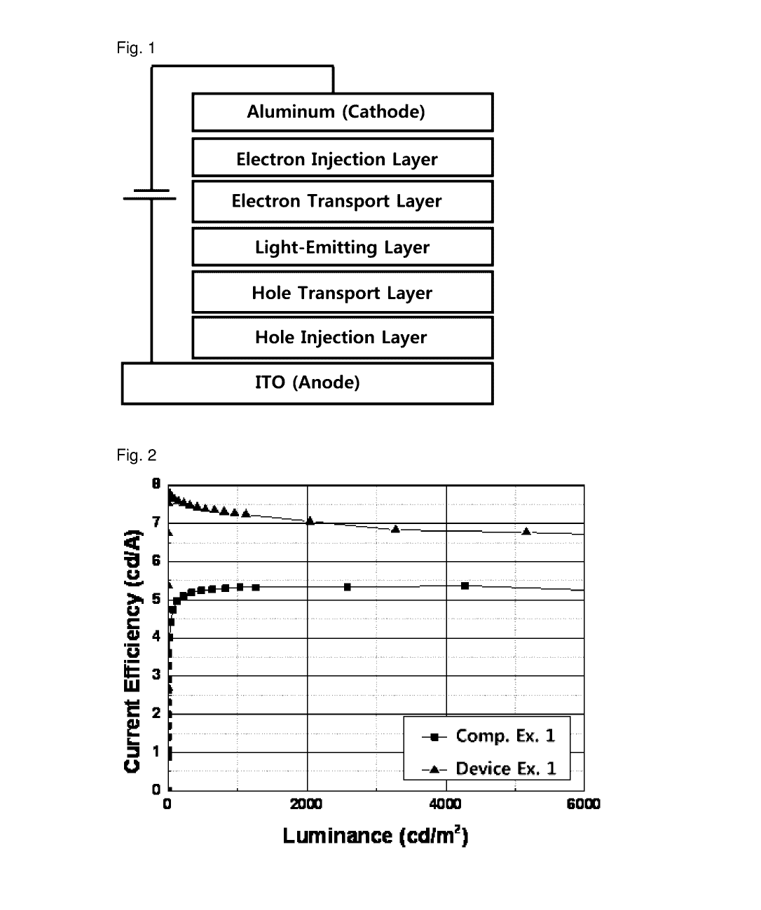

FIG. 1 illustrates a schematic sectional view of an organic electroluminescent device comprising the electron transport layer comprising the electron transport material according to one embodiment of the present invention.

The organic EL device according to the present invention comprises an anode; a cathode; and at least one organic layer between the two electrodes, wherein the organic layer comprises a light-emitting layer which contains a host and a dopant. The light-emitting layer emits light, which may be a single layer or multi-layers having two or more layers. The doping concentration of the dopant compound to the host compound in the light-emitting layer is preferably less than 20 wt %.

The organic EL device of the present invention may comprise an electron transport material in the organic layer and use a reductive dopant as a dopant of the light-emitting layer. The reductive dopant is one or more selected from the group consisting of alkali metals, alkaline-earth metals, rare-earth metals, alkali metal oxides, alkali metal halides, alkaline-earth metal oxides, alkaline-earth metal halides, rare-earth metal oxides, rare-earth metal halides, organic complexes of an alkali metal, organic complexes of an alkaline-earth metal, and organic complexes of a rare-earth metal.

The organic EL device of the present invention may further include at least one compound selected from the group consisting of arylamine-based compounds and styrylarylamine-based compounds in the organic layer.

In the organic EL device of the present invention, an organic layer may further comprise at least one metal selected from the group consisting of metals of Group 1, metals of Group 2, transition metals of the 4^{th} period, transition metals of the 5^{th} period, lanthanides, and organic metals of d-transition elements of the Periodic Table, or at least one complex compound comprising the metal.

Preferably, in the organic EL device of the present invention, at least one layer (hereinafter, “a surface layer”) selected from a chalcogenide layer, a metal halide layer, and a metal oxide layer may be placed on an inner surface(s) of one or both electrode(s). Specifically, it is preferred that a chalcogenide (including oxides) layer of silicon or aluminum is placed on an anode surface of a light-emitting medium layer, and a metal halide layer or metal oxide layer is placed on a cathode surface of an electroluminescent medium layer. The surface layer provides operating stability for the organic EL device. Preferably, the chalcogenide includes SiOₓ (1≦X≦2), AlOₓ (1≦X≦1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer (HIL), a hole transport layer (HTL), an electron blocking layer (EBL), or their combinations can be used between the anode and the light-emitting layer. The hole injection layer may be multi-layers in order to lower the hole injection barrier (or hole injection voltage) from the anode to the hole transport layer or the electron blocking layer, wherein each of the multi-layers simultaneously may use two compounds. The hole transport layer or the electron blocking layer may also be multi-layers.

An electron buffer layer, a hole blocking layer (HBL), an electron transport layer (ETL), an electron injection layer (EIL), or their combinations can be used between the light-emitting layer and the cathode. The electron buffer layer may be multi-layers in order to control the injection of the electron and improve the interface properties between the light-emitting layer and the electron injection layer, wherein each of the multi-layers simultaneously may use two compounds. The hole blocking layer or the electron transport layer may also be multi-layers, wherein each of the multi-layers may use a multi-component of compounds.

Preferably, in the organic EL device of the present invention, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to prepare an organic EL device having two or more light-emitting layers and emitting white light.

In order to form each layer constituting the organic EL device of the present invention, dry film-forming methods such as vacuum deposition, sputtering, plasma, ion plating methods, etc., or wet film-forming methods such as spin coating, dip coating, flow coating methods, etc., can be used.

When using a wet film-forming method, a thin film is formed by dissolving or dispersing the material constituting each layer in suitable solvents, such as ethanol, chloroform, tetrahydrofuran, dioxane, etc. The solvents are not specifically limited as long as the material constituting each layer is soluble or dispersible in the solvents, which do not cause any problems in forming a layer.

Hereinafter, the compounds of the present invention, the preparation method thereof, and luminous properties of devices comprising the electron transport material comprising the compound will be explained in detail with reference to the following examples.

### EXAMPLE 1

### Preparation of Compound ETL-75

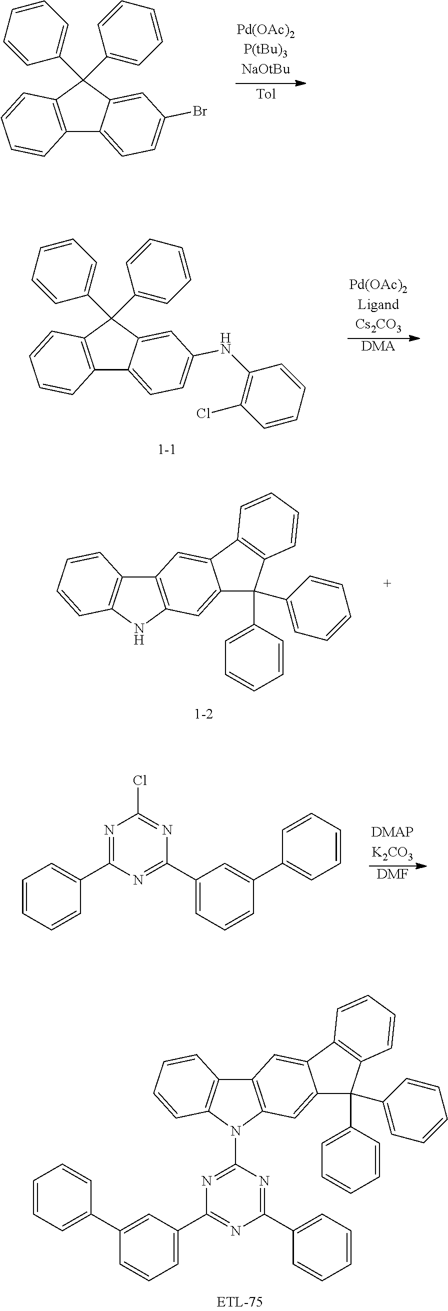

### Preparation of Compound 1-1

After introducing 2-bromo-9,9-diphenyl-9H-fluorene (8 g, 0.020 mol), 2-chloroaniline (3.1 mL, 0.030 mol), Pd(OAc)₂ (181 mg, 0.805 mol), P(t-Bu)₃ (50%) (0.8 mL, 1.61 mmol), NaOt-Bu (4.8 g, 0.056 mol), and toluene 58 mL in a flask, the mixture was stirred at 140° C. for 4 hours. After the reaction, the mixture was washed with distilled water, and an organic layer was extracted with ethylacetate (EA). The organic layer was then dried with MgSO₄, the solvent was removed with a rotary evaporator, and the remaining product was purified with column chromatography to obtain compound 1-1 (7.3 g, 82%).

### Preparation of Compound 1-2

After introducing compound 1-1 (7.3 g, 0.016 mol) in a flask, Pd(OAc)₂ (190 mg, 0.84 mmol), tricyclohexylphosphonium tetrafluoroborate (620 mg, 0.0016 mol), Cs₂CO₃ (16 g, 0.050 mol), and dimethylacetamide (DMA) 85 mL were added to the mixture. The reactant mixture was heated to 190° C. and stirred for 5 hours. After the reaction, the mixture was washed with distilled water, and an organic layer was extracted with EA. The organic layer was then dried with MgSO₄, the solvent was removed with a rotary evaporator, and the remaining product was purified with column chromatography to obtain compound 1-2 (4.8 g, 59%).

### Preparation of Compound ETL-75

After introducing compound 1-2 (4.8 g, 0.011 mol) in a flask, 2-([1,1′-biphenyl]-3-yl)-4-chloro-6-phenyl-1,3,5-triazine (4.8 g, 0.014 mol), dimethylaminopyridine (DMAP) (720 mg, 0.005 mmol), K₂CO₃ (4.0 g, 0.029 mol), and dimethylformamide (DMF) 120 mL were added to the mixture. The reactant mixture was heated to 120° C. and stirred for 3 hours. After the reaction, the mixture was washed with distilled water, and an organic layer was extracted with EA. The organic layer was then dried with MgSO₄, the solvent was removed with a rotary evaporator, and the remaining product was purified with column chromatography to obtain compound ETL-75 (6.9 g, 82%).

Compounds ETL-1 to ETL-86 were prepared using the same synthetic method of Example 1. Among them, specific physical property data of the representative compounds are shown in Table 1 as follows:

TABLE 1

| | | | UV | | |
|---|---|---|---|---|---|
| | | | Spectrum | PL Spectrum | |
| | | | (nm, in | (nm, in | |
| Compound | Yield (%) | MP (° C.) | toluene) | toluene) | Mass |
| ETL-21 | 65 | 253 | 354 | 480 | 564 |
| ETL-28 | 60 | 250 | 334 | 428 | 680 |
| ETL-30 | 36 | 299 | 332 | 386 | 805 |
| ETL-31 | 60 | 212 | 368 | 433 | 640 |
| ETL-32 | 31 | 289 | 384 | 436 | 690 |
| ETL-33 | 76 | 266 | 370 | 489 | 614 |
| ETL-34 | 70 | 255 | 356 | 521 | 564 |
| ETL-35 | 12 | 218 | 358 | 445 | 640 |
| ETL-36 | 67 | 261 | 344 | 521 | 614 |
| ETL-46 | 89 | 277 | 336 | 481 | 578 |
| ETL-47 | 50 | 243 | 332 | 424 | 654 |
| ETL-52 | 38 | 280 | 346 | 484 | 564 |
| ETL-53 | 60 | 289 | 344 | 479 | 685 |
| ETL-54 | 27 | 240 | 308 | 451 | 590.7 |
| ETL-60 | 47 | 301 | 344 | 483 | 653 |
| ETL-61 | 35 | 289 | 372 | 479 | 670 |
| ETL-66 | 37 | 321 | 384 | 491 | 640 |
| ETL-67 | 22 | 235 | 336 | 521 | 668 |
| ETL-68 | 47 | 298 | 376 | 482 | 563 |
| ETL-70 | 52 | 337 | 310 | 464 | 714.9 |
| ETL-71 | 49 | 256 | 372 | 487 | 614 |
| ETL-74 | 55 | 340 | 324 | 484 | 640 |
| ETL-75 | 47 | 326 | 334 | 486 | 714 |
| ETL-76 | 47 | 382 | 361 (MC) | 514 (MC) | 790 |
| ETL-77 | 23 | 198 | 258 (MC) | 535 (MC) | 590.00 |
| ETL-80 | 83 | 387 | 258 (MC) | 535 (MC) | 640.00 |
| ETL-81 | 29 | 371 | 257 (MC) | 543 (MC) | 744.80 |
| ETL-82 | 57 | 363 | 238 (MC) | 532 (MC) | 728.00 |
| ETL-83 | 25 | 289.0 | 310.0 | 498.0 | 511.73 |
| ETL-84 | 80 | 397 | 282 (MC) | 533 (MC) | 714.00 |
| ETL-85 | 30 | 267 | 254 (MC) | 493 (MC) | 713.00 |

[MC is methylenechloride]

### DEVICE EXAMPLE 1

### Production of an OLED Device Comprising the Electron Transport Material According to the Present Invention

An OLED device was produced using the electron transport material of the present invention. A transparent electrode indium tin oxide (ITO) thin film (15 Ω/sq) on a glass substrate for an OLED device (Geomatec Co. LTD., Japan) was subjected to an ultrasonic washing with trichloroethylene, acetone, ethanol, and distilled water, sequentially, and was then stored in isopropanol. The ITO substrate was then mounted on a substrate holder of a vacuum vapor depositing apparatus. N⁴,N^{4′}-diphenyl-N⁴,N^{4′}-bis(9-phenyl-9H-carbazol-3-yl)-[1,1′-biphenyl]-4,4′-diamine was introduced into a cell of the vacuum vapor depositing apparatus, and the pressure in the chamber of the apparatus was then controlled to 10⁻⁶ torr. Thereafter, an electric current was applied to the cell to evaporate the introduced material, thereby forming hole injection layer 1 having a thickness of 60 nm on the ITO substrate. 1,4,5,8,9,12-hexaazatriphenylene-hexacarbonitrile was then introduced into another cell of the vacuum vapor depositing apparatus, and an electric current was applied to the cell to evaporate the introduced material, thereby forming hole injection layer 2 having a thickness of 5 nm on hole injection layer 1. N-([1,1′-biphenyl]-4-yl)-9,9-dimethyl-N-(4-(9-phenyl-9H-carbazol-3-yl)phenyl)-9H-fluorene-2-amine was introduced into one cell of the vacuum vapor depositing apparatus. Thereafter, an electric current was applied to the cell to evaporate the introduced material, thereby forming hole transport layer 1 having a thickness of 20 nm on hole injection layer 2. N,N-di([1,1′-biphenyl]-4-yl)-4′-(9H-carbazol-9-yl)-[1,1′-biphenyl]-4-amine was then introduced into another cell of the vacuum vapor depositing apparatus, and an electric current was applied to the cell to evaporate the introduced material, thereby forming hole transport layer 2 having a thickness of 5 nm on hole transport layer 1. Thereafter, BH-1 as a host was introduced into one cell of the vacuum vapor depositing apparatus and BD-1 as a dopant was introduced into another cell. The two materials were evaporated at a different rate and the dopant was deposited in a doping amount of 2 wt %, based on the total weight of the host and dopant, to form a light-emitting layer having a thickness of 20 nm on the hole transport layer. Compound ETL-75 was then evaporated on one cell to form an electron transport layer having a thickness of 35 nm on the light-emitting layer. After depositing lithium quinolate having a thickness of 4 nm as an electron injection layer on the electron transport layer, an Al cathode having a thickness of 80 nm was then deposited by another vacuum vapor deposition apparatus on the electron injection layer. Thus, an OLED device was produced. All the materials used for producing the OLED device were purified by vacuum sublimation at 10⁻⁶ torr prior to use.

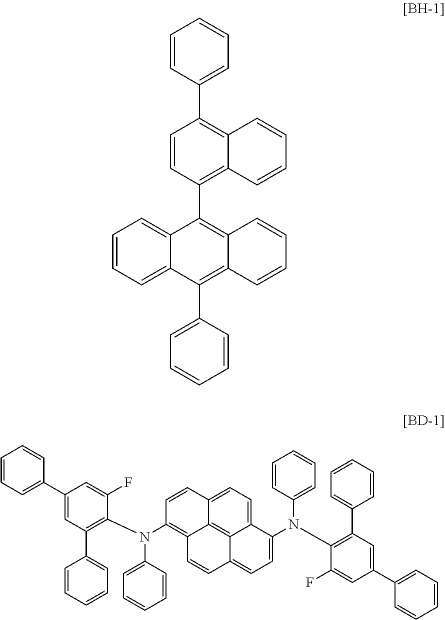

### DEVICE EXAMPLE 2

### Production of an OLED Device Comprising the Electron Transport Material According to the Present Invention

An OLED device was produced in the same manner as in Device Example 1, except that compound ETL-78 was used in the electron transport layer.

### Device Example 3

### Production of an OLED Device Comprising the Electron Transport Material According to the Present Invention

An OLED device was produced in the same manner as in Device Example 1, except that compound ETL-80 was used in the electron transport layer.

### DEVICE EXAMPLE 4

### Production of an OLED Device Comprising the Electron Transport Material According to the Present Invention

An OLED device was produced in the same manner as in Device Example 1, except that compound ETL-84 was used in the electron transport layer.

### COMPARATIVE EXAMPLE 1

### Production of an OLED Device Comprising a Conventional Electron Transport Material

An OLED device was produced in the same manner as in Device Example 1, except that the following comparative compound was used in the electron transport layer.

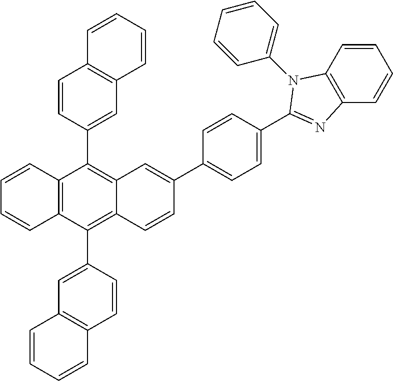 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

The current efficiencies according to luminance values of the OLED devices produced above are shown in FIG. 2.

Furthermore, driving voltage at a luminance of 1,000 nit, luminous efficiency, CIE color coordinate, and the time taken for the luminance at 2,000 nit to be reduced from 100% to 90% at a constant current of the OLEDs produced as above are shown in Table 2 below.

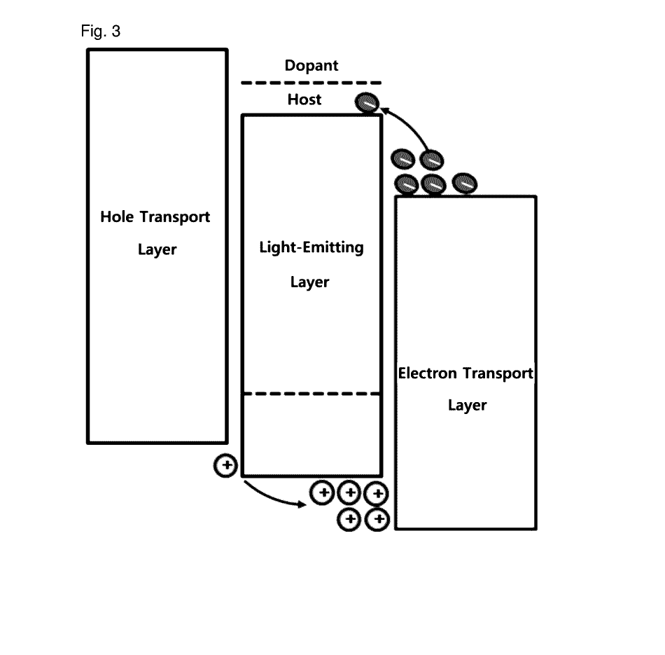

The data of the above Device Examples 1 to 4 and Comparative Example 1 were determined under the condition of ┌ electron affinity of the electron transport layer (Ab)>electron affinity of the host (Ah)┘. The electron transport layers of Device Examples 1 to 4 have higher LUMO (lowest unoccupied molecular orbital) than that of Comparative Example 1. As depicted in FIG. 3, the devices according to the present invention have a large barrier between the light-emitting layer and the electron transport layer in the process of transporting electrons compared with the device of Comparative Example 1. However, in accordance with the fast electron current characteristic of the diphenyl structure, the devices of the present invention have lower driving voltage and higher efficiency than the device of Comparative Example 1.

LUMO energy value and HOMO (highest occupied molecular orbital) energy value have inherently negative numbers, but LUMO energy value and HOMO energy value in the present invention are conveniently expressed in their absolute values. Furthermore, the comparison between LUMO energy values is based on their absolute values. LUMO energy value and HOMO energy value in the present invention are calculated by Density Functional Theory (DFT).

TABLE 2

| | Electron | | | Color | Color | | | |
|---|---|---|---|---|---|---|---|---|
| | Transport | Voltage | Efficiency | Coordinate | Coordinate | Lifespan | LUMO | HOMO |
| | Layer | (V) | (cd/A) | (x) | (y) | (hr) | (eV) | (eV) |
| Device Ex. 1 | ETL-75 | 4.3 | 7.2 | 0.138 | 0.103 | 36.2 | 1.90 | 5.43 |
| Device Ex. 2 | ETL-78 | 4.4 | 7.1 | 0.138 | 0.100 | 26.5 | 1.89 | 5.40 |
| Device Ex. 3 | ETL-80 | 4.2 | 7.7 | 0.138 | 0.105 | 24.4 | 1.88 | 5.43 |
| Device Ex. 4 | ETL-84 | 4.3 | 7.6 | 0.138 | 0.105 | 27.6 | 1.90 | 5.42 |
| Comp. Ex. 1 | Comparative | 4.9 | 5.3 | 0.141 | 0.134 | 23.1 | 1.81 | 5.12 |
| | Compound | | | | | | | |

The organic electroluminescent compound of the present invention has lower driving voltage, higher efficiency, and longer lifespan than the conventional material.

In addition, the movement of excitons produced in the light-emitting layer and hole carriers are efficiently restricted as shown in FIG. 3. According to this, the compound of the present invention is regarded as showing color coordinates being the closest to pure blue compared with the comparative compound of Comparative Example 1.

Comparison of Electron Current Characteristic of the Comparative Compound and the Compound of the Present Invention

In order to demonstrate fast electron current characteristic of the electron transport layer in the devices according to the present invention, voltage property was compared by preparing an Electron Only Device (EOD).

The device was produced as follows: Barium, 2,9-dimethyl-4,7-diphenyl-1,10-phenanthroline (BCP) were introduced into a cell in a vacuum vapor depositing apparatus. Thereafter, an electric current was applied to the cell to evaporate the introduced materials, thereby forming a hole blocking layer (HBL) having a thickness of 10 nm on the ITO substrate. Next, BH-1 as a host was introduced into one cell of the vacuum vapor depositing apparatus, and BD-1 as a dopant was introduced into another cell. The two materials were evaporated at a different rate and the dopant was deposited in a doping amount of 2 wt %, based on the total weight of the host and dopant, to form a light-emitting layer having a thickness of 20 nm on a hole transport layer. The compounds in the table below were then evaporated to form an electron transport layer having a thickness of 33 nm on the light-emitting layer. After depositing lithium quinolate having a thickness of 4 nm as an electron injection layer on the electron transport layer, an Al cathode having a thickness of 80 nm was then deposited by another vacuum vapor deposition apparatus on the electron injection layer. Thus, an OLED device was produced. All the materials used for producing the OLED device were purified by vacuum sublimation at 10⁻⁶ torr prior to use. Voltages at 10 and 50 mA/cm² according to each electron transport material are shown in Table 3 below.

TABLE 3

| Electron | Voltage (V) | Voltage (V) |
|---|---|---|
| Transport Layer | (10 mA/cm²) | (50 mA/cm²) |
| Comparative | 4.5 | 5.1 |
| Compound | | |
| ETL-75 | 3.6 | 4.9 |
| ETL-78 | 3.7 | 4.9 |
| ETL-80 | 3.4 | 4.6 |
| ETL-84 | 3.4 | 4.7 |

As shown in Table 3 above, the compounds of the present invention have faster electron current characteristics at both voltages (10 and 50 mA/cm²) than the comparative compound. The EOD verified that the compounds of the present invention were suitable to provide low driving voltage and high efficiency of the device.

1. An electron transport material comprising a compound represented by the following formula 1: 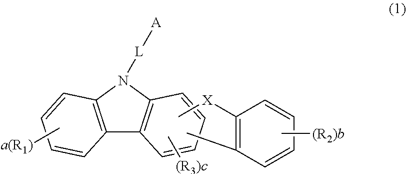
wherein
A represents a substituted or unsubstituted 5- to 30-membered heteroaryl;
L represents a single bond, a substituted or unsubstituted (C6-C30)arylene, or a substituted or unsubstituted 5- to 30-membered heteroarylene;
X represents CR₁₁R₁₂;
R₁ and R₂ each independently represent hydrogen, deuterium, a halogen, a cyano, a substituted or unsubstituted (C1-C30)alkyl, a substituted or unsubstituted (C6-C30)aryl, a substituted or unsubstituted 5- to 30-membered heteroaryl, a substituted or unsubstituted (C6-C30)aryl(C1-C30)alkyl, a substituted or unsubstituted (C3-C30)cycloalkyl, a substituted or unsubstituted (C1-C30)alkoxy, a substituted or unsubstituted (C1-C30)alkylsilyl, a substituted or unsubstituted (C6-C30)arylsilyl, a substituted or unsubstituted (C6-C30)aryl(C1-C30)alkylsilyl, a substituted or unsubstituted (C1-C30)alkylamino, a substituted or unsubstituted (C6-C30)arylamino, or a substituted or unsubstituted (C1-C30)alkyl(C6-C30)arylamino; or are linked to an adjacent substituent(s) to form a mono- or polycyclic (C3-C30) alicyclic or aromatic ring whose carbon atom(s) may be replaced with at least one hetero atom selected from nitrogen, oxygen, and sulfur;
R₃ represents hydrogen, deuterium, a halogen, a cyano, a substituted or unsubstituted (C1-C30)alkyl, a substituted or unsubstituted (C6-C30)aryl, or a substituted or unsubstituted 5- to 30-membered heteroaryl; or are linked to an adjacent substituent(s) to form a mono- or polycyclic (C3-C30) alicyclic or aromatic ring whose carbon atom(s) may be replaced with at least one hetero atom selected from nitrogen, oxygen, and sulfur;
R₁₁ and R₁₂ each independently represent a substituted or unsubstituted (C1-C30)alkyl, a substituted or unsubstituted (C6-C30)aryl, or a substituted or unsubstituted 5- to 30-membered heteroaryl; or are linked to each other to form a mono- or polycyclic (C3-C30) alicyclic or aromatic ring whose carbon atom(s) may be replaced with at least one hetero atom selected from nitrogen, oxygen, and sulfur;
a and b each independently represent an integer of 1 to 4, where a or b is an integer of 2 or more, each of R₁ and each of R₂ may be the same or different;
c represents an integer of 1 to 2, where c is 2, each of R₃ may be the same or different; and
the heteroaryl(ene) contains at least one hetero atom selected from B, N, O, S, P(═O), Si, and P.

2. The electron transport material according to claim 1, wherein formula 1 is represented by one of the following formulae 2 to 7: 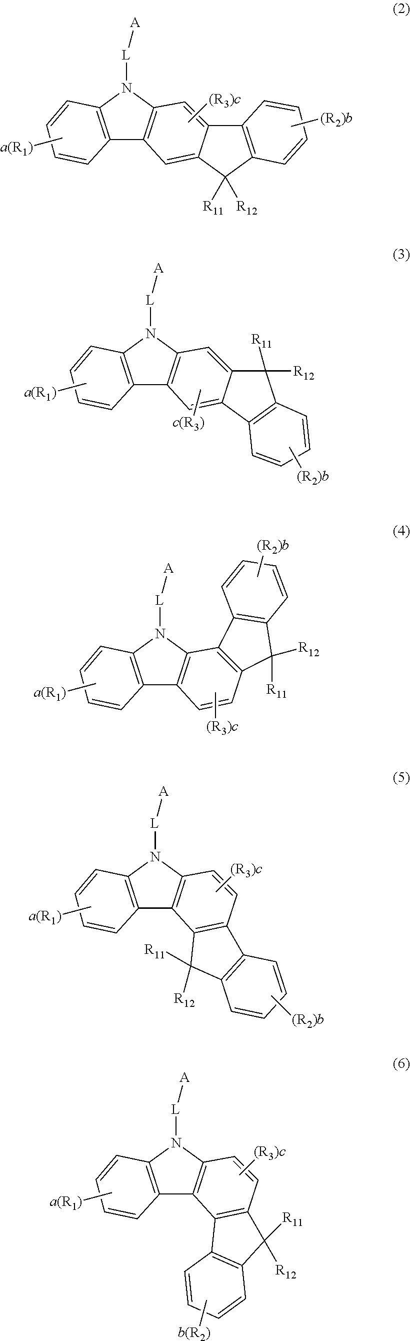 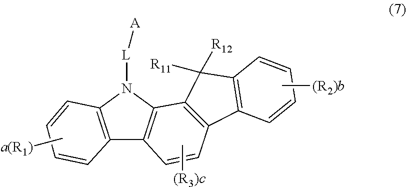
wherein A, L, R₁ to R₃, R₁₁, R₁₂, a, b, and c are as defined in claim 1.

3. The electron transport material according to claim 1, wherein the substituents of the substituted alkyl, the substituted alkoxy, the substituted cycloalkyl, the substituted aryl(ene), the substituted heteroaryl(ene), the substituted alkylsilyl, the substituted arylsilyl, the substituted arylalkylsilyl, the substituted arylamino, the substituted alkylamino, the substituted alkylarylamino, and the substituted arylalkyl in A, L, R₁ to R₃, R₁₁, and R₁₂ each independently are at least one selected from the group consisting of deuterium, a halogen, a cyano, a carboxyl, a nitro, a hydroxyl, a (C1-C30)alkyl, a halo(C1-C30)alkyl, a (C2-C30)alkenyl, a (C2-C30)alkynyl, a (C1-C30)alkoxy, a (C1-C30)alkylthio, a (C3-C30)cycloalkyl, a (C3-C30)cycloalkenyl, a 3- to 7-membered heterocycloalkyl, a (C6-C30)aryloxy, a (C6-C30)arylthio, a 3- to 30-membered heteroaryl unsubstituted or substituted with a (C6-C30)aryl, a (C6-C30)aryl, a (C6-C30)aryl substituted with a 3- to 30-membered heteroaryl, a (C6-C30)aryl substituted with a tri(C1-C30)alkylsilyl, a (C6-C30)aryl substituted with a tri(C6-C30)arylsilyl, a tri(C1-C30)alkylsilyl, a tri(C6-C30)arylsilyl, a di(C1-C30)alkyl(C6-C30)arylsilyl, a (C1-C30)alkyldi(C6-C30)arylsilyl, an amino, a mono- or di-(C1-C30)alkylamino, a mono- or di-(C6-C30)arylamino, a (C1-C30)alkyl(C6-C30)arylamino, a (C1-C30)alkylcarbonyl, a (C1-C30)alkoxycarbonyl, a (C6-C30)arylcarbonyl, a di(C6-C30)arylboronyl, a di(C1-C30)alkylboronyl, a (C1-C30)alkyl(C6-C30)arylboronyl, a (C6-C30)aryl(C1-C30)alkyl, and a (C1-C30)alkyl(C6-C30)aryl.

4. The electron transport material according to claim 1, wherein
A represents a substituted or unsubstituted 5- to 20-membered heteroaryl;
L represents a single bond, a substituted or unsubstituted (C6-C20)arylene, or a substituted or unsubstituted 5- to 20-membered heteroarylene;
X represents CR₁₁R₁₂;
R₁ and R₂ each independently represent hydrogen, a substituted or unsubstituted (C6-C20)aryl, or a substituted or unsubstituted 5- to 20-membered heteroaryl;
R₃ represents hydrogen;
R₁₁ and R₁₂ each independently represent a substituted or unsubstituted (C1-C6)alkyl, or a substituted or unsubstituted (C6-C20)aryl; or are linked to each other to form a mono- or polycyclic (C5-C20) alicyclic or aromatic ring;
a and b each independently represent an integer of 1 to 2; and
c represents 1.

5. The electron transport material according to claim 1, wherein
A represents an unsubstituted 5- to 20-membered heteroaryl, a 5- to 20-membered heteroaryl substituted with a 5- to 20-membered heteroaryl unsubstituted or substituted with a (C6-C20)aryl, a 5- to 20-membered heteroaryl substituted with a (C6-C20)aryl, a 5- to 20-membered heteroaryl substituted with a (06-C20)aryl substituted with a 5- to 20-membered heteroaryl, a 5- to 20-membered heteroaryl substituted with a (C6-C20)aryl substituted with a tri(C1-C6)alkylsilyl, a 5- to 20-membered heteroaryl substituted with a (C6-C20)aryl substituted with a tri(C6-C20)arylsilyl, or a 5- to 20-membered heteroaryl substituted with a (C1-C6)alkyl(C6-C20)aryl;
L represents a single bond, an unsubstituted (C6-C20)arylene, or an unsubstituted 5- to 20-membered heteroarylene;
X represents CR₁₁R₁₂;
R₁ and R₂ each independently represent hydrogen, a (C6-C20)aryl unsubstituted or substituted with a (C6-C12)aryl, or a 5- to 20-membered heteroaryl unsubstituted or substituted with a (C6-C20)aryl;
R₃ represents hydrogen;
R₁₁ and R₁₂ each independently represent an unsubstituted (C1-C6)alkyl, or an unsubstituted (C6-C20)aryl; or are linked to each other to form a mono- or polycyclic (C5-C20) alicyclic or aromatic ring;
a and b each independently represent an integer of 1 to 2; and
c represents 1.

6. The electron transport material according to claim 1, wherein A represents a substituted or unsubstituted pyridine, a substituted or unsubstituted pyrimidine, a substituted or unsubstituted triazine, a substituted or unsubstituted pyrazine, a substituted or unsubstituted quinoline, a substituted or unsubstituted quinazoline, a substituted or unsubstituted quinoxaline, a substituted or unsubstituted naphthyridine, or a substituted or unsubstituted phenanthroline.

7. The electron transport material according to claim 1, wherein the compound represented by formula 1 is selected from the group consisting of:

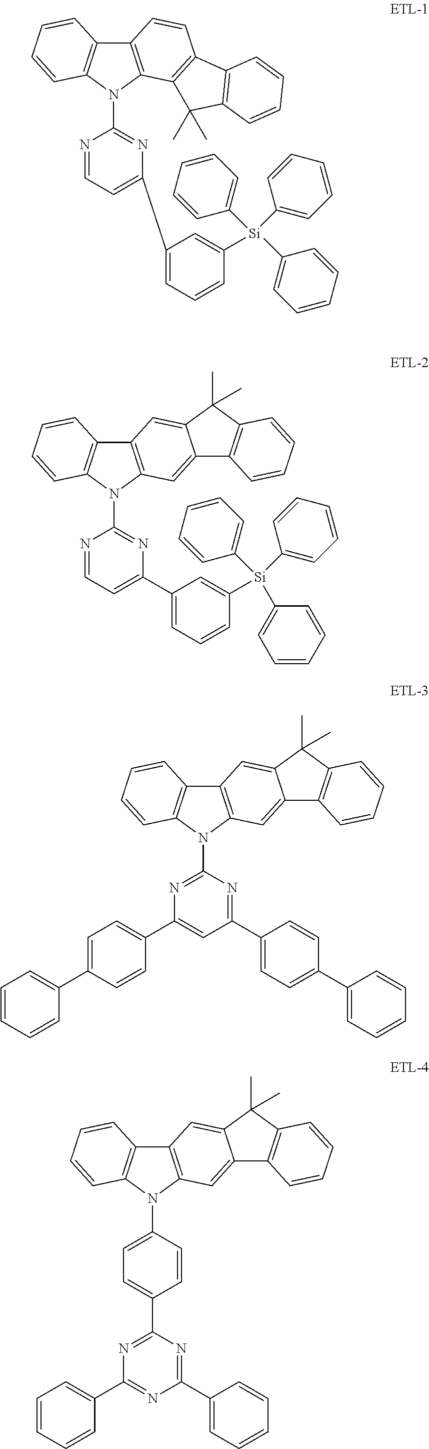 [SMILES: CC1(C)c2ccccc2-c2ccc3c4ccccc4n(-c4nccc(-c5cccc([Si](c6ccccc6)(c6ccccc6)c6ccccc6)c5)n4)c3c21 ; CC1(C)c2ccccc2-c2cc3c(cc21)c1ccccc1n3-c1nccc(-c2cccc([Si](c3ccccc3)(c3ccccc3)c3ccccc3)c2)n1 ; CC1(C)c2ccccc2-c2cc3c(cc21)c1ccccc1n3-c1nc(-c2ccc(-c3ccccc3)cc2)cc(-c2ccc(-c3ccccc3)cc2)n1 ; CC1(C)c2ccccc2-c2cc3c(cc21)c1ccccc1n3-c1ccc(-c2nc(-c3ccccc3)nc(-c3ccccc3)n2)cc1]

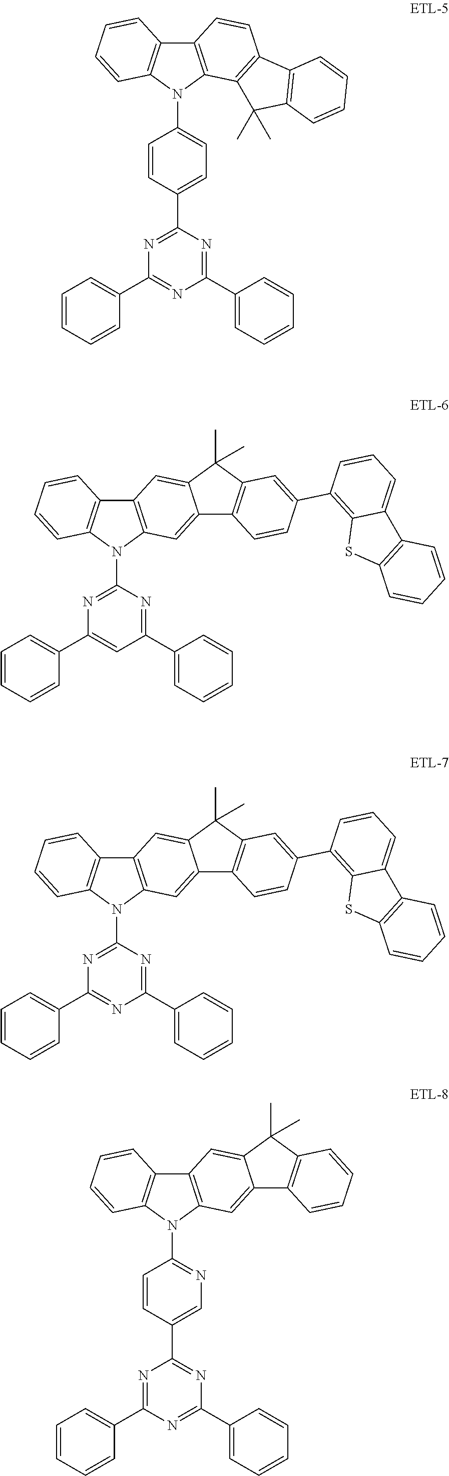 [SMILES: CC1(C)c2ccccc2-c2ccc3c4ccccc4n(-c4ccc(-c5nc(-c6ccccc6)nc(-c6ccccc6)n5)cc4)c3c21 ; CC1(C)c2cc(-c3cccc4c3sc3ccccc34)ccc2-c2cc3c(cc21)c1ccccc1n3-c1nc(-c2ccccc2)cc(-c2ccccc2)n1 ; CC1(C)c2cc(-c3cccc4c3sc3ccccc34)ccc2-c2cc3c(cc21)c1ccccc1n3-c1nc(-c2ccccc2)nc(-c2ccccc2)n1 ; CC1(C)c2ccccc2-c2cc3c(cc21)c1ccccc1n3-c1ccc(-c2nc(-c3ccccc3)nc(-c3ccccc3)n2)cn1]

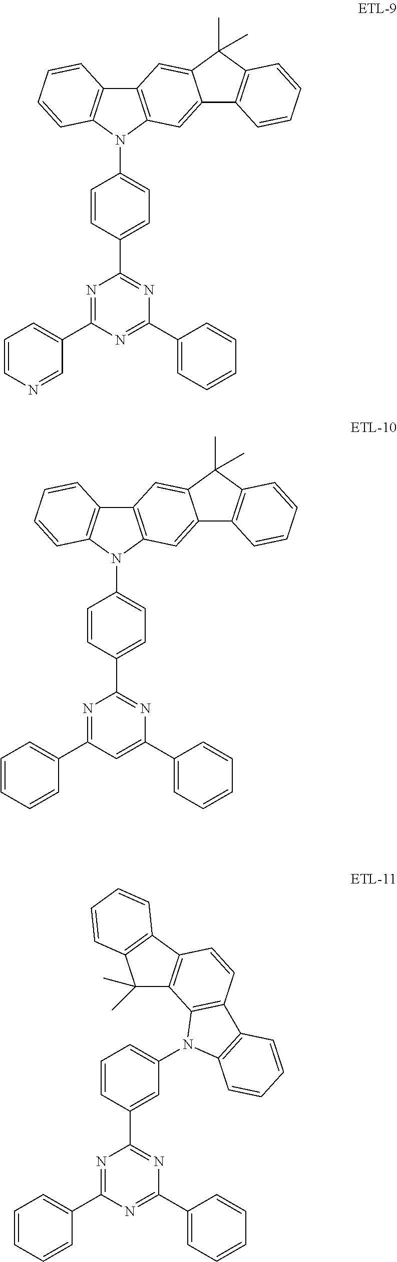 [SMILES: CC1(C)c2ccccc2-c2cc3c(cc21)c1ccccc1n3-c1ccc(-c2nc(-c3ccccc3)nc(-c3cccnc3)n2)cc1 ; CC1(C)c2ccccc2-c2cc3c(cc21)c1ccccc1n3-c1ccc(-c2nc(-c3ccccc3)cc(-c3ccccc3)n2)cc1 ; CC1(C)c2ccccc2-c2ccc3c4ccccc4n(-c4cccc(-c5nc(-c6ccccc6)nc(-c6ccccc6)n5)c4)c3c21]

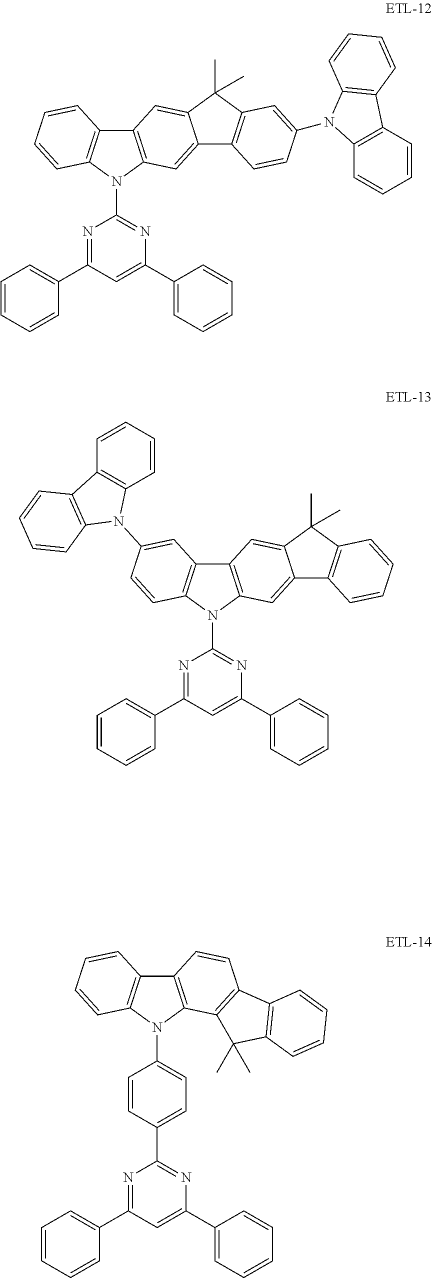 [SMILES: CC1(C)c2cc(-n3c4ccccc4c4ccccc43)ccc2-c2cc3c(cc21)c1ccccc1n3-c1nc(-c2ccccc2)cc(-c2ccccc2)n1 ; CC1(C)c2ccccc2-c2cc3c(cc21)c1cc(-n2c4ccccc4c4ccccc42)ccc1n3-c1nc(-c2ccccc2)cc(-c2ccccc2)n1 ; CC1(C)c2ccccc2-c2ccc3c4ccccc4n(-c4ccc(-c5nc(-c6ccccc6)cc(-c6ccccc6)n5)cc4)c3c21]

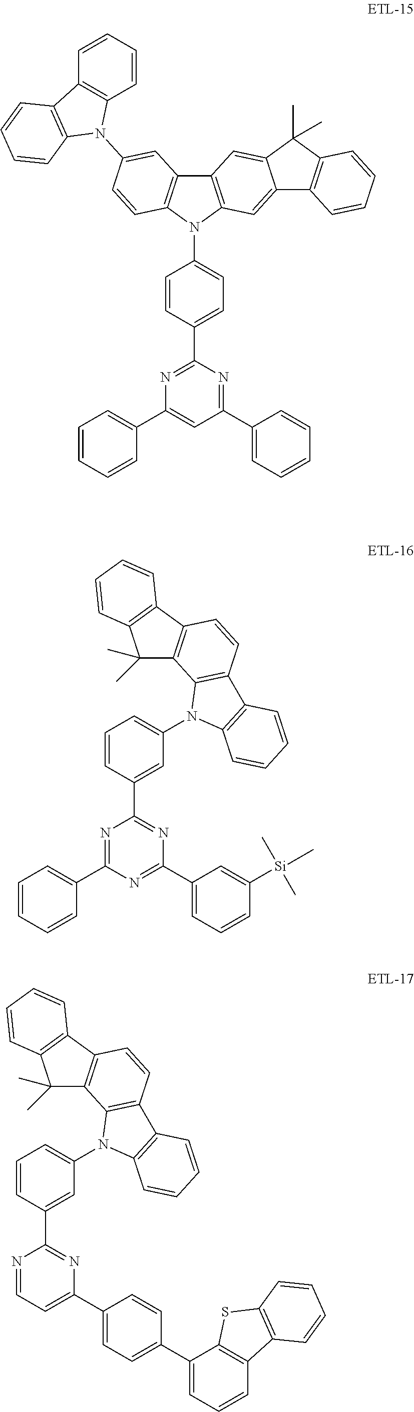 [SMILES: CC1(C)c2ccccc2-c2cc3c(cc21)c1cc(-n2c4ccccc4c4ccccc42)ccc1n3-c1ccc(-c2nc(-c3ccccc3)cc(-c3ccccc3)n2)cc1 ; CC1(C)c2ccccc2-c2ccc3c4ccccc4n(-c4cccc(-c5nc(-c6ccccc6)nc(-c6cccc([Si](C)(C)C)c6)n5)c4)c3c21 ; CC1(C)c2ccccc2-c2ccc3c4ccccc4n(-c4cccc(-c5nccc(-c6ccc(-c7cccc8c7sc7ccccc78)cc6)n5)c4)c3c21]

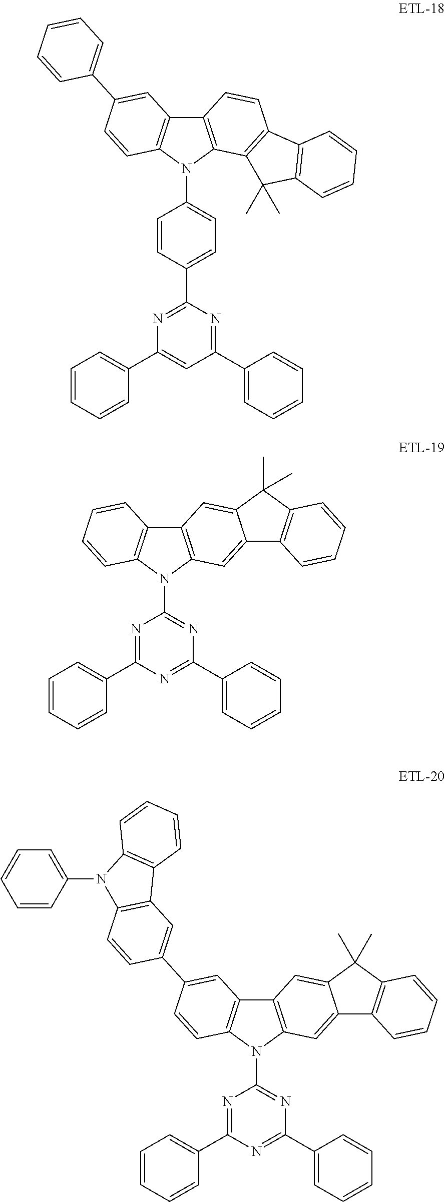

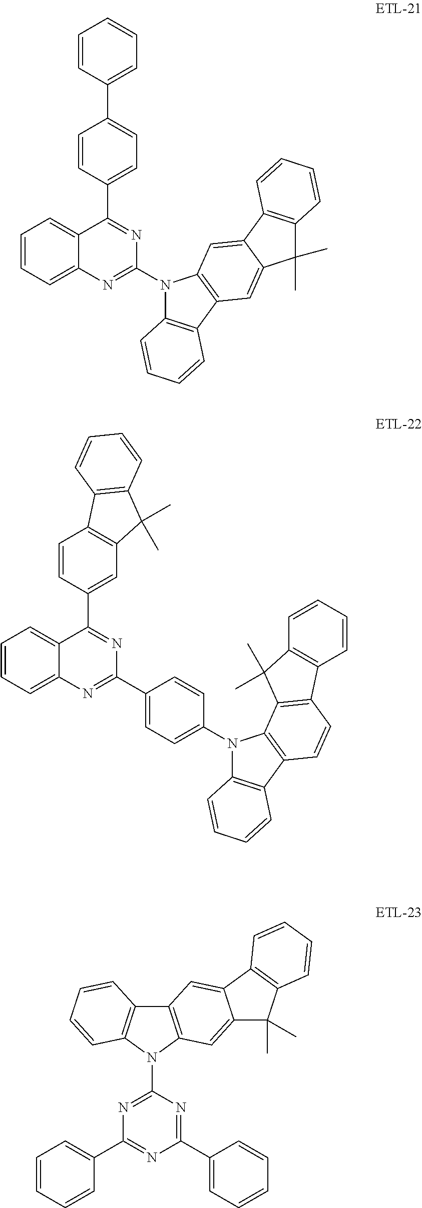 [SMILES: CC1(C)c2ccccc2-c2cc3c(cc21)c1ccccc1n3-c1nc(-c2ccc(-c3ccccc3)cc2)c2ccccc2n1 ; CC1(C)c2ccccc2-c2ccc(-c3nc(-c4ccc(-n5c6ccccc6c6ccc7c(c65)C(C)(C)c5ccccc5-7)cc4)nc4ccccc34)cc21 ; CC1(C)c2ccccc2-c2cc3c4ccccc4n(-c4nc(-c5ccccc5)nc(-c5ccccc5)n4)c3cc21]

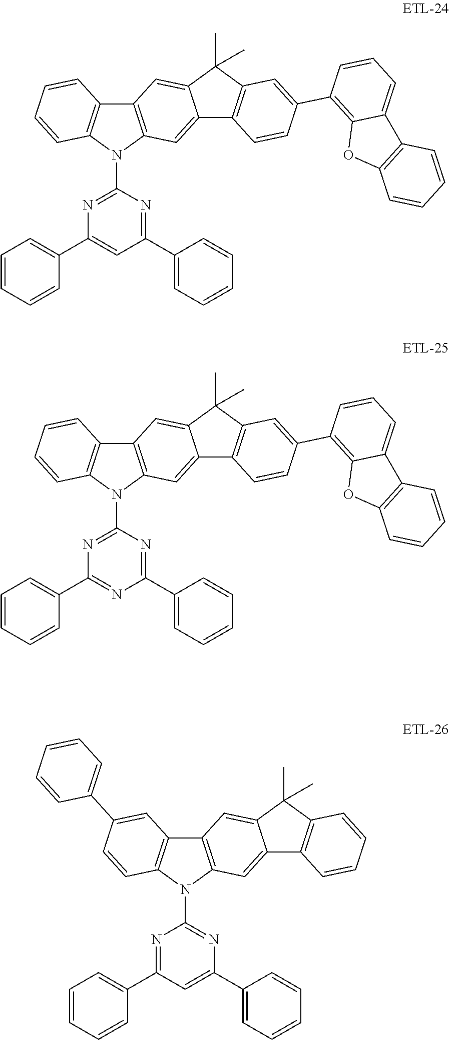 [SMILES: CC1(C)c2cc(-c3cccc4c3oc3ccccc34)ccc2-c2cc3c(cc21)c1ccccc1n3-c1nc(-c2ccccc2)cc(-c2ccccc2)n1 ; CC1(C)c2cc(-c3cccc4c3oc3ccccc34)ccc2-c2cc3c(cc21)c1ccccc1n3-c1nc(-c2ccccc2)nc(-c2ccccc2)n1 ; CC1(C)c2ccccc2-c2cc3c(cc21)c1cc(-c2ccccc2)ccc1n3-c1nc(-c2ccccc2)cc(-c2ccccc2)n1]

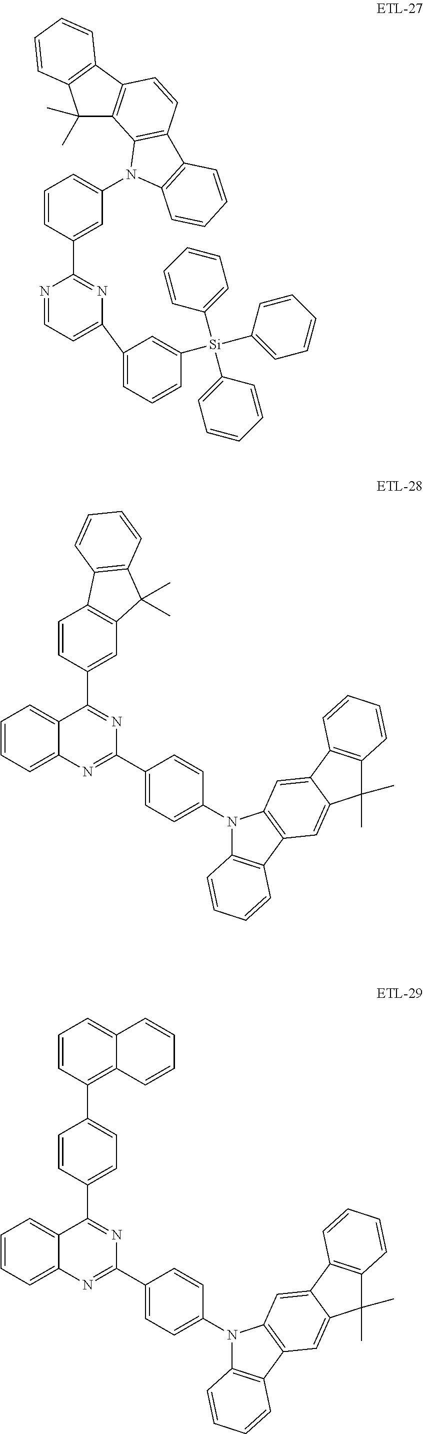 [SMILES: CC1(C)c2ccccc2-c2ccc3c4ccccc4n(-c4cccc(-c5nccc(-c6cccc([Si](c7ccccc7)(c7ccccc7)c7ccccc7)c6)n5)c4)c3c21 ; CC1(C)c2ccccc2-c2ccc(-c3nc(-c4ccc(-n5c6ccccc6c6cc7c(cc65)-c5ccccc5C7(C)C)cc4)nc4ccccc34)cc21 ; CC1(C)c2ccccc2-c2cc3c(cc21)c1ccccc1n3-c1ccc(-c2nc(-c3ccc(-c4cccc5ccccc45)cc3)c3ccccc3n2)cc1]

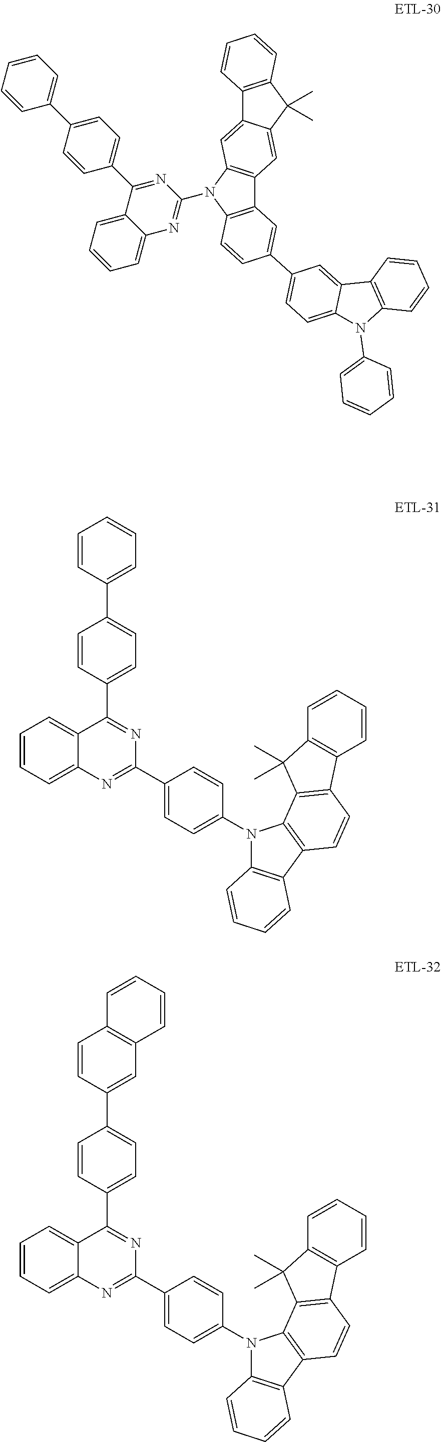 [SMILES: CC1(C)c2ccccc2-c2cc3c(cc21)c1cc(-c2ccc4c(c2)c2ccccc2n4-c2ccccc2)ccc1n3-c1nc(-c2ccc(-c3ccccc3)cc2)c2ccccc2n1 ; CC1(C)c2ccccc2-c2ccc3c4ccccc4n(-c4ccc(-c5nc(-c6ccc(-c7ccccc7)cc6)c6ccccc6n5)cc4)c3c21 ; CC1(C)c2ccccc2-c2ccc3c4ccccc4n(-c4ccc(-c5nc(-c6ccc(-c7ccc8ccccc8c7)cc6)c6ccccc6n5)cc4)c3c21]

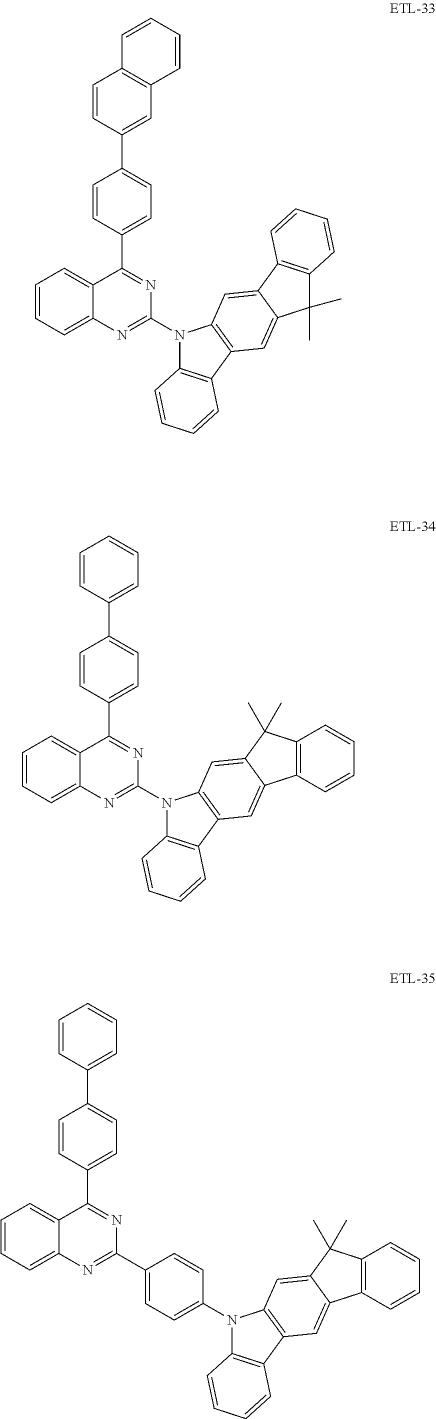 [SMILES: CC1(C)c2ccccc2-c2cc3c(cc21)c1ccccc1n3-c1nc(-c2ccc(-c3ccc4ccccc4c3)cc2)c2ccccc2n1 ; CC1(C)c2ccccc2-c2cc3c4ccccc4n(-c4nc(-c5ccc(-c6ccccc6)cc5)c5ccccc5n4)c3cc21 ; CC1(C)c2ccccc2-c2cc3c4ccccc4n(-c4ccc(-c5nc(-c6ccc(-c7ccccc7)cc6)c6ccccc6n5)cc4)c3cc21]

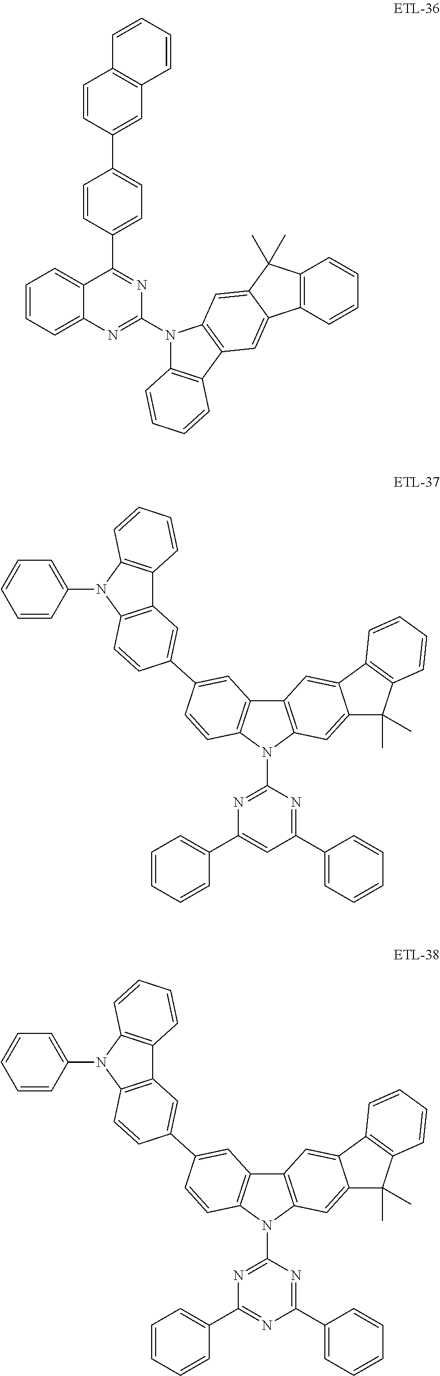 [SMILES: CC1(C)c2ccccc2-c2cc3c4ccccc4n(-c4nc(-c5ccc(-c6ccc7ccccc7c6)cc5)c5ccccc5n4)c3cc21 ; CC1(C)c2ccccc2-c2cc3c4cc(-c5ccc6c(c5)c5ccccc5n6-c5ccccc5)ccc4n(-c4nc(-c5ccccc5)cc(-c5ccccc5)n4)c3cc21 ; CC1(C)c2ccccc2-c2cc3c4cc(-c5ccc6c(c5)c5ccccc5n6-c5ccccc5)ccc4n(-c4nc(-c5ccccc5)nc(-c5ccccc5)n4)c3cc21]

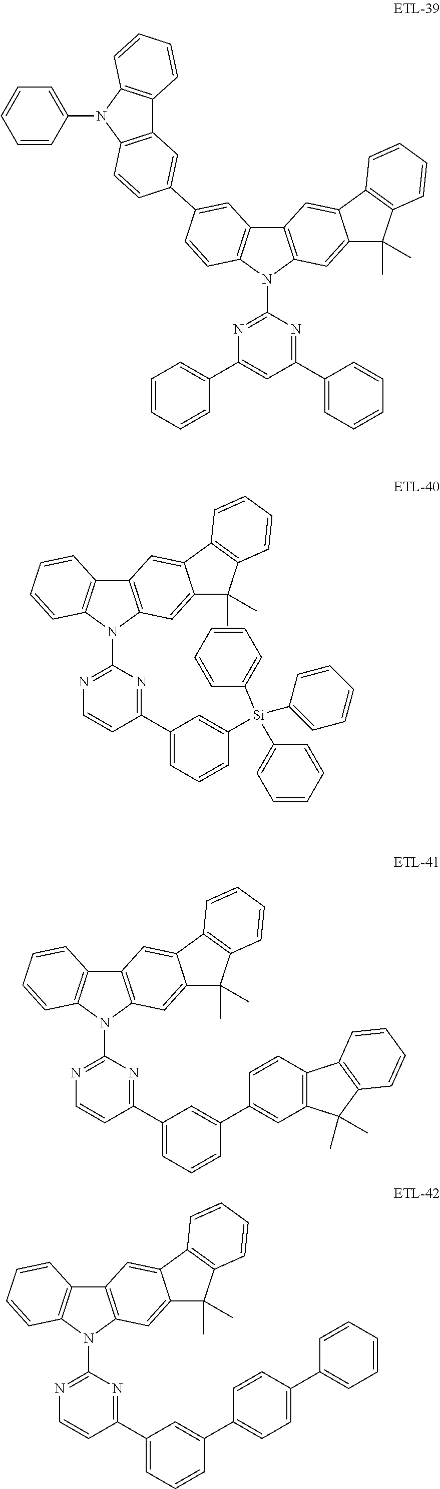

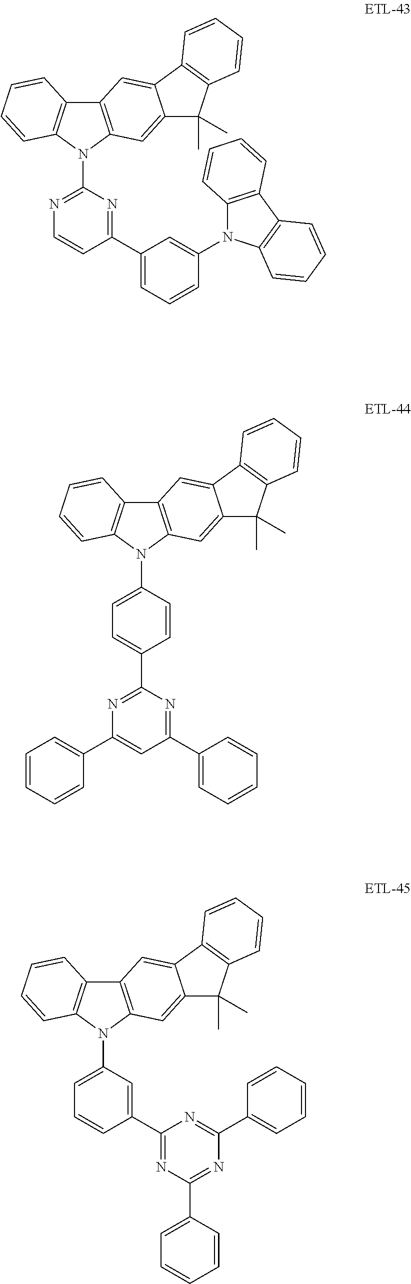 [SMILES: CC1(C)c2ccccc2-c2cc3c4ccccc4n(-c4nccc(-c5cccc(-n6c7ccccc7c7ccccc76)c5)n4)c3cc21 ; CC1(C)c2ccccc2-c2cc3c4ccccc4n(-c4ccc(-c5nc(-c6ccccc6)cc(-c6ccccc6)n5)cc4)c3cc21 ; CC1(C)c2ccccc2-c2cc3c4ccccc4n(-c4cccc(-c5nc(-c6ccccc6)nc(-c6ccccc6)n5)c4)c3cc21]

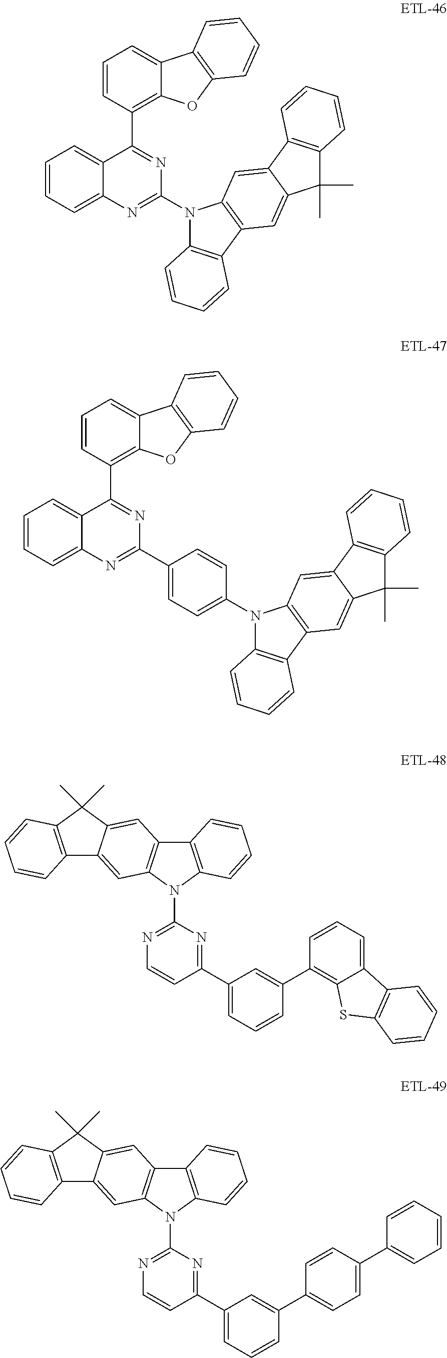 [SMILES: CC1(C)c2ccccc2-c2cc3c(cc21)c1ccccc1n3-c1nc(-c2cccc3c2oc2ccccc23)c2ccccc2n1 ; CC1(C)c2ccccc2-c2cc3c(cc21)c1ccccc1n3-c1ccc(-c2nc(-c3cccc4c3oc3ccccc34)c3ccccc3n2)cc1 ; CC1(C)c2ccccc2-c2cc3c(cc21)c1ccccc1n3-c1nccc(-c2cccc(-c3cccc4c3sc3ccccc34)c2)n1 ; CC1(C)c2ccccc2-c2cc3c(cc21)c1ccccc1n3-c1nccc(-c2cccc(-c3ccc(-c4ccccc4)cc3)c2)n1]

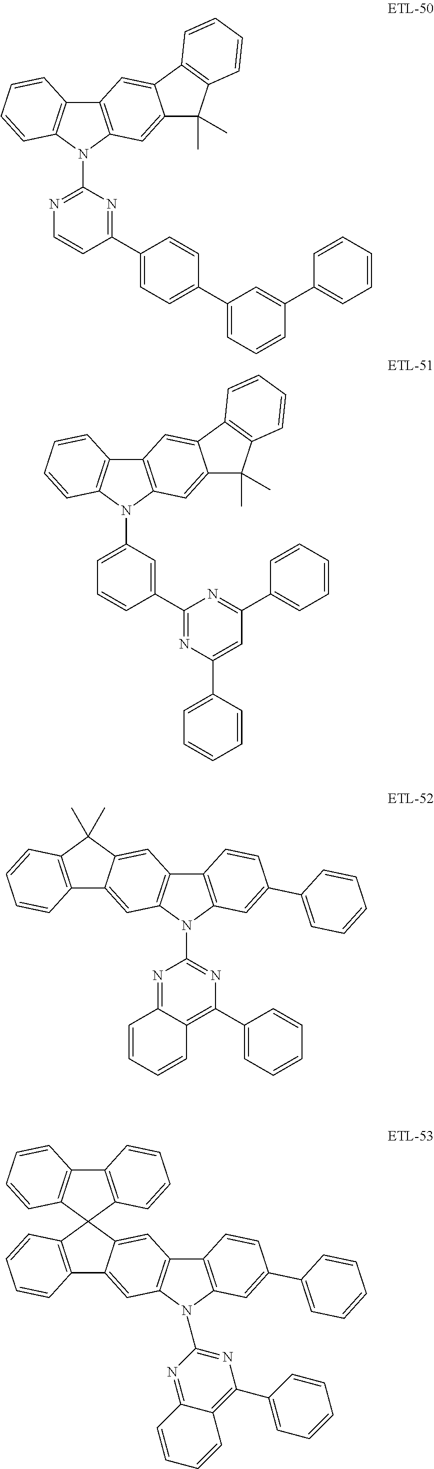

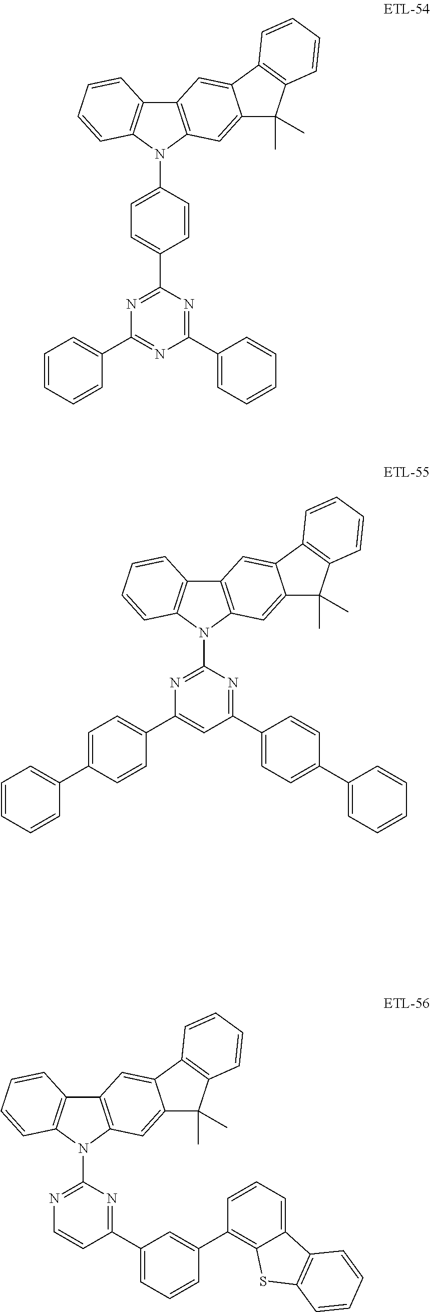 [SMILES: CC1(C)c2ccccc2-c2cc3c4ccccc4n(-c4ccc(-c5nc(-c6ccccc6)nc(-c6ccccc6)n5)cc4)c3cc21 ; CC1(C)c2ccccc2-c2cc3c4ccccc4n(-c4nc(-c5ccc(-c6ccccc6)cc5)cc(-c5ccc(-c6ccccc6)cc5)n4)c3cc21 ; CC1(C)c2ccccc2-c2cc3c4ccccc4n(-c4nccc(-c5cccc(-c6cccc7c6sc6ccccc67)c5)n4)c3cc21]

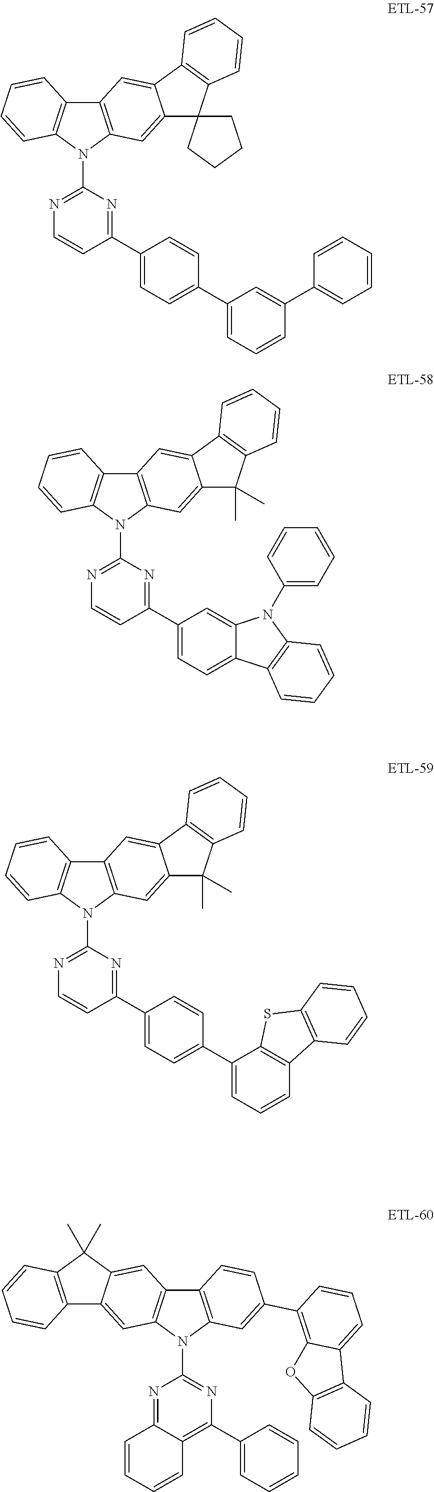

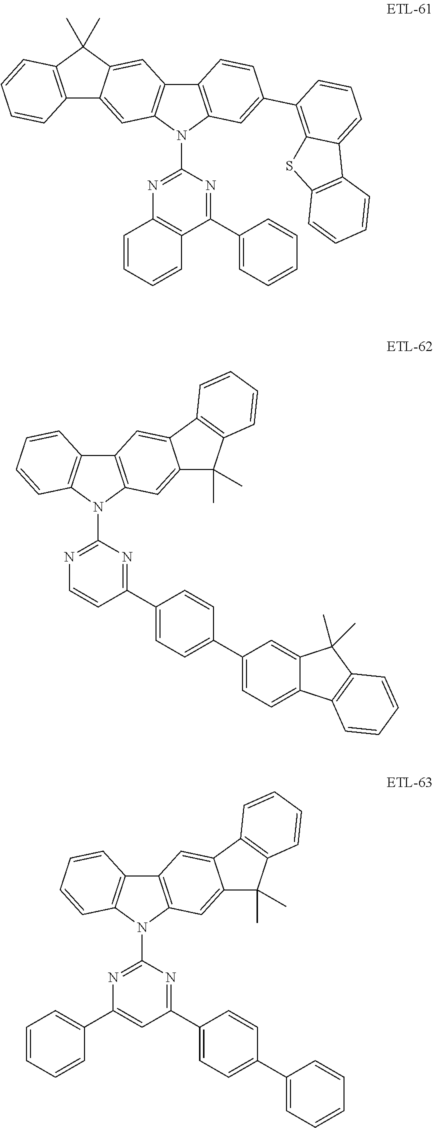 [SMILES: CC1(C)c2ccccc2-c2cc3c(cc21)c1ccc(-c2cccc4c2sc2ccccc24)cc1n3-c1nc(-c2ccccc2)c2ccccc2n1 ; CC1(C)c2ccccc2-c2ccc(-c3ccc(-c4ccnc(-n5c6ccccc6c6cc7c(cc65)C(C)(C)c5ccccc5-7)n4)cc3)cc21 ; CC1(C)c2ccccc2-c2cc3c4ccccc4n(-c4nc(-c5ccccc5)cc(-c5ccc(-c6ccccc6)cc5)n4)c3cc21]

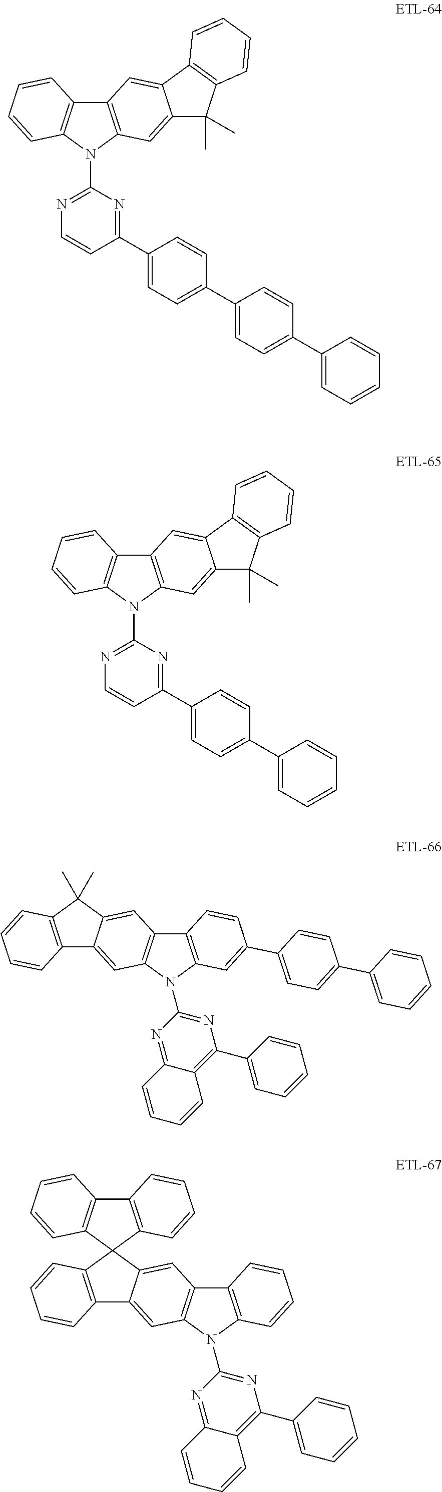 [SMILES: CC1(C)c2ccccc2-c2cc3c4ccccc4n(-c4nccc(-c5ccc(-c6ccc(-c7ccccc7)cc6)cc5)n4)c3cc21 ; CC1(C)c2ccccc2-c2cc3c4ccccc4n(-c4nccc(-c5ccc(-c6ccccc6)cc5)n4)c3cc21 ; CC1(C)c2ccccc2-c2cc3c(cc21)c1ccc(-c2ccc(-c4ccccc4)cc2)cc1n3-c1nc(-c2ccccc2)c2ccccc2n1 ; c1ccc(-c2nc(-n3c4ccccc4c4cc5c(cc43)-c3ccccc3C53c4ccccc4-c4ccccc43)nc3ccccc23)cc1]

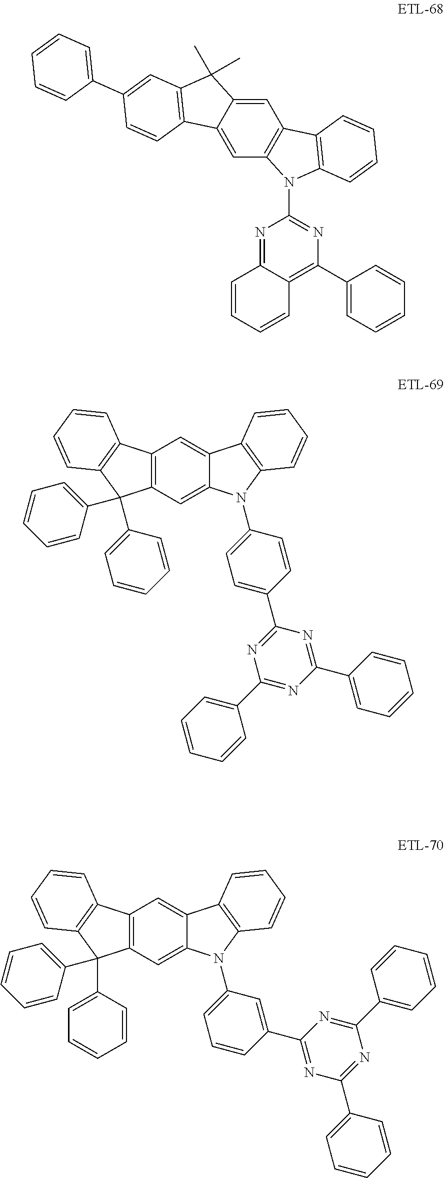 [SMILES: CC1(C)c2cc(-c3ccccc3)ccc2-c2cc3c(cc21)c1ccccc1n3-c1nc(-c2ccccc2)c2ccccc2n1 ; c1ccc(-c2nc(-c3ccccc3)nc(-c3ccc(-n4c5ccccc5c5cc6c(cc54)C(c4ccccc4)(c4ccccc4)c4ccccc4-6)cc3)n2)cc1 ; c1ccc(-c2nc(-c3ccccc3)nc(-c3cccc(-n4c5ccccc5c5cc6c(cc54)C(c4ccccc4)(c4ccccc4)c4ccccc4-6)c3)n2)cc1]

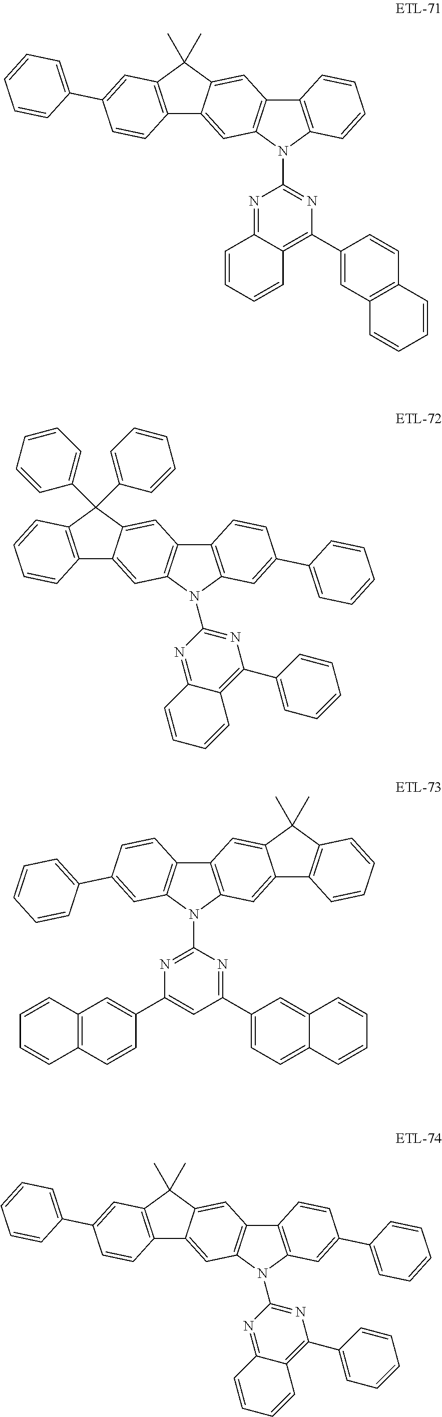 [SMILES: CC1(C)c2cc(-c3ccccc3)ccc2-c2cc3c(cc21)c1ccccc1n3-c1nc(-c2ccc3ccccc3c2)c2ccccc2n1 ; c1ccc(-c2ccc3c4cc5c(cc4n(-c4nc(-c6ccccc6)c6ccccc6n4)c3c2)-c2ccccc2C5(c2ccccc2)c2ccccc2)cc1 ; CC1(C)c2ccccc2-c2cc3c(cc21)c1ccc(-c2ccccc2)cc1n3-c1nc(-c2ccc3ccccc3c2)cc(-c2ccc3ccccc3c2)n1 ; CC1(C)c2cc(-c3ccccc3)ccc2-c2cc3c(cc21)c1ccc(-c2ccccc2)cc1n3-c1nc(-c2ccccc2)c2ccccc2n1]

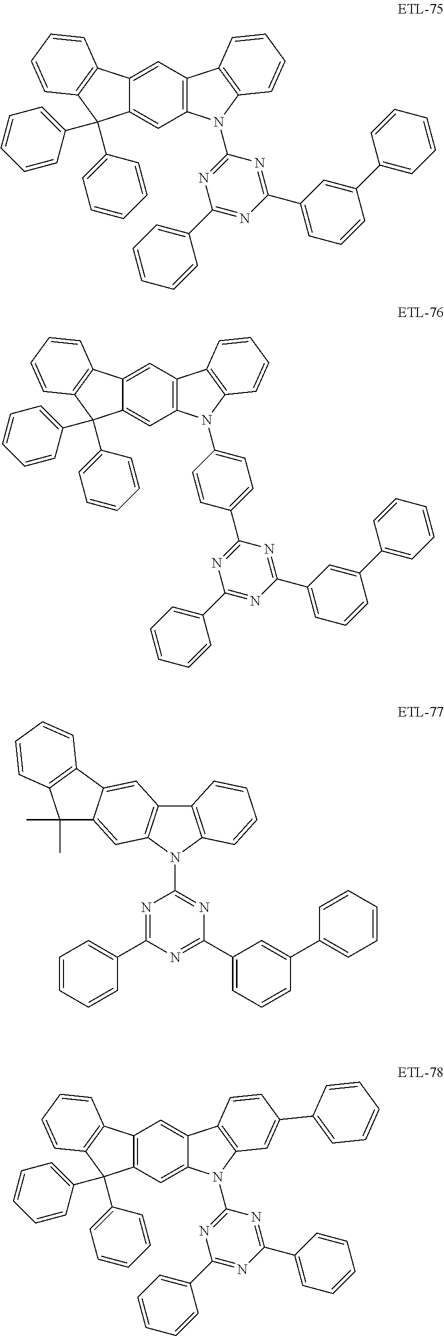 [SMILES: c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; c1ccc(-c2cccc(-c3nc(-c4ccccc4)nc(-c4ccc(-n5c6ccccc6c6cc7c(cc65)C(c5ccccc5)(c5ccccc5)c5ccccc5-7)cc4)n3)c2)cc1 ; CC1(C)c2ccccc2-c2cc3c4ccccc4n(-c4nc(-c5ccccc5)nc(-c5cccc(-c6ccccc6)c5)n4)c3cc21 ; c1ccc(-c2ccc3c4cc5c(cc4n(-c4nc(-c6ccccc6)nc(-c6ccccc6)n4)c3c2)C(c2ccccc2)(c2ccccc2)c2ccccc2-5)cc1]

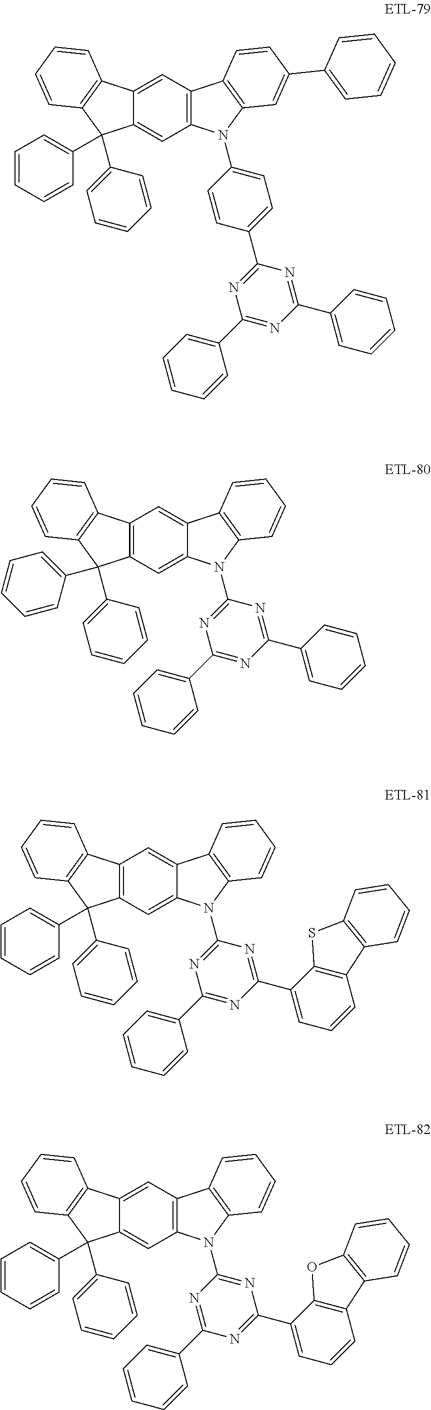 [SMILES: c1ccc(-c2ccc3c4cc5c(cc4n(-c4ccc(-c6nc(-c7ccccc7)nc(-c7ccccc7)n6)cc4)c3c2)C(c2ccccc2)(c2ccccc2)c2ccccc2-5)cc1 ; c1ccc(-c2nc(-c3ccccc3)nc(-n3c4ccccc4c4cc5c(cc43)C(c3ccccc3)(c3ccccc3)c3ccccc3-5)n2)cc1 ; c1ccc(-c2nc(-c3cccc4c3sc3ccccc34)nc(-n3c4ccccc4c4cc5c(cc43)C(c3ccccc3)(c3ccccc3)c3ccccc3-5)n2)cc1 ; c1ccc(-c2nc(-c3cccc4c3oc3ccccc34)nc(-n3c4ccccc4c4cc5c(cc43)C(c3ccccc3)(c3ccccc3)c3ccccc3-5)n2)cc1]

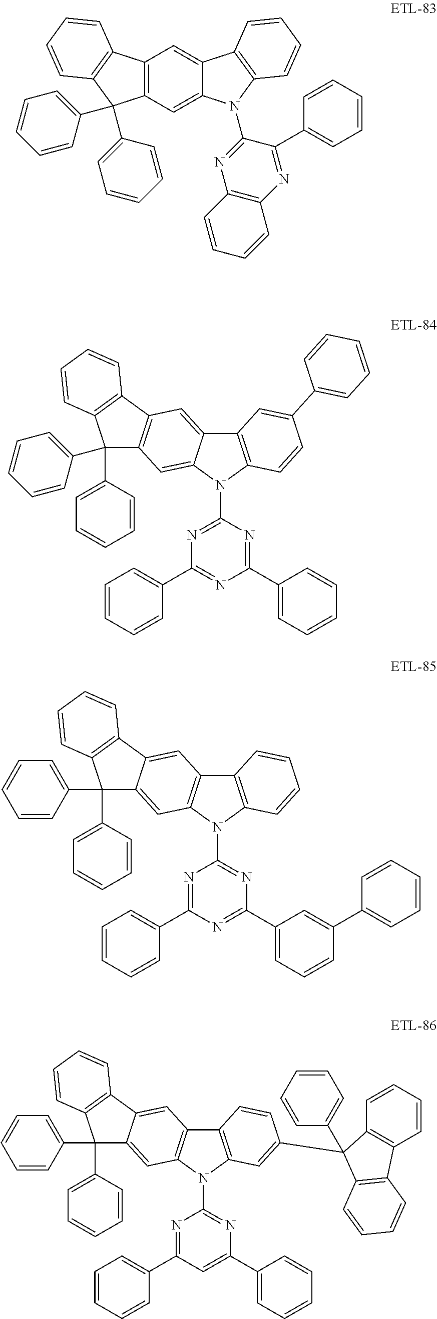 [SMILES: c1ccc(-c2nc3ccccc3nc2-n2c3ccccc3c3cc4c(cc32)C(c2ccccc2)(c2ccccc2)c2ccccc2-4)cc1 ; c1ccc(-c2ccc3c(c2)c2cc4c(cc2n3-c2nc(-c3ccccc3)nc(-c3ccccc3)n2)C(c2ccccc2)(c2ccccc2)c2ccccc2-4)cc1 ; c1ccc(-c2cccc(-c3nc(-c4ccccc4)nc(-n4c5ccccc5c5cc6c(cc54)C(c4ccccc4)(c4ccccc4)c4ccccc4-6)n3)c2)cc1 ; c1ccc(-c2cc(-c3ccccc3)nc(-n3c4cc(C5(c6ccccc6)c6ccccc6-c6ccccc65)ccc4c4cc5c(cc43)C(c3ccccc3)(c3ccccc3)c3ccccc3-5)n2)cc1]

8. An organic electroluminescent device comprising the electron transport material according to claim 1.

9. The organic electroluminescent device according to claim 8, further comprising a reductive dopant.

10. The organic electroluminescent device according to claim 9, wherein the reductive dopant is at least one selected from the group consisting of alkali metals, alkaline-earth metals, rare-earth metals, alkali metal oxides, alkali metal halides, alkaline-earth metal oxides, alkaline-earth metal halides, rare-earth metal oxides, rare-earth metal halides, organic complexes of an alkali metal, organic complexes of an alkaline-earth metal, and organic complexes of a rare-earth metal.
